(19) United States
(12) Patent Application Publication
(10) Pub. No.: US 2023/0210755 A1
(43) Pub. Date: Jul. 6, 2023

(54) FUNCTIONAL SKIN COATING POLYMER

(71) Applicant: The Johns Hopkins University, Baltimore, MD (US)

(72) Inventors: Sewon Kang, Baltimore, MD (US); Adam W. Freeman, Baltimore, MD (US)

(21) Appl. No.: 17/998,839

(22) PCT Filed: May 27, 2021
(86) PCT No.: PCT/US21/34446

§ 371 Date: Nov. 15, 2022

Related U.S. Application Data
(60) Provisional application No. 63/030,491, filed on May 27, 2020.

(51) Int. Cl. A61K 8/895; A61Q 17/04; C08G 77/12; C08G 77/20; C08G 77/38; C08G 77/388; A61K 8/898

(52) U.S. Cl. CPC A61K 8/895; A61Q 17/04; C08G 77/12; C08G 77/20; C08G 77/38; C08G 77/388; A61K 8/898; A61K 2800/88

(57) ABSTRACT

Film-forming polymers that contain covalently-attached or non-covalently bound light-filtering, e.g., UV-absorbing, compounds and their use as a skin-protectant coating, such as a sunscreen, are disclosed.

### BACKGROUND

Human beings require protection from the sun to prevent sunburn and premature aging and to mitigate the skin cancer risks associated with exposure to damaging solar radiation. Historically, such protection has been accomplished through the use of protective structures, parasols, and clothing. Since the late 1920s, however, this arsenal has been augmented with a variety of creams and lotions, which are applied directly to the skin and contain ingredients that absorb or reflect the incoming radiation, thereby attenuating the amount of incoming radiation reaching the skin and limiting its harmful effects. Shaath, 2011. In subsequent years, these sunscreens and sunblocks, which are often used as first line of defense against harmful solar radiation, have evolved to contain an ever-broadening range of molecules that perform that task more efficiently and over a broader range of the ultraviolet (UV) spectrum (FIG. 1).

Representative organic molecules commonly used in sunscreen formulations are shown in FIG. 2A-2D. In spite of their great structural diversity, they function similarly, absorbing incoming UV light and converting it into heat through a variety of structurally-dependent relaxation pathways. The photostability of each organic UV absorber, which is the time period over which it can perform that task successfully under constant irradiation, ranges from a few minutes to several hours. At that point, the molecular structure of the organic UV absorber will have changed and/or degraded such that it no longer absorbs the incoming UV rays. This limited stability, together with the fact that they can be washed and sweated off of the skin, collectively require frequent reapplication of sunscreens to maintain efficacy. Further, many published studies have shown that an overwhelming majority of people fail to apply enough sunscreen, resulting in inadequate protection.

In addition to the aforementioned issue with application and adherence, many of the organic absorbers shown in FIG. 2A-2D, although they perform their intended function well when applied correctly, have unfortunate downsides. Since they are small organic molecules, which are often made more lipophilic via derivatization with fatty alkyl chains to improve formulation compatibility and wash-off resistance, they and their degradation products can readily diffuse into the upper layers of the skin, which serves as a conduit to the body. Matta et al., 2019; The Trouble with Ingredients in Sunscreens. There, they variably act as photosensitizers, endocrine disruptors, and allergens. Wang et al, 2016. More troubling, several of the organic absorbers cross into the human blood stream and breastmilk, where they can cause widespread systemic problems and be passed on to breastfed children. Schlumpf et al., 2010.

In addition to the unintended consequences of organic UV absorbers on the human body, several of the most commonly used UV absorbers also have been shown to negatively affect the environment. In 2015, a marine conservation organization estimated that approximately 14,000 tons of sunscreen ended up in coral reefs around the world annually. Downs et al., 2016; Safe Suncreen Council. Subsequent studies revealed that octinoxate and oxybenzone significantly harm marine environments and ecosystems, including coral reefs. In fact, oxybenzone has been linked to deformities in coral larvae and both chemicals have been implicated in coral bleaching, a devastating process in which coral loses symbiotic algae. These findings have led the Hawaii legislature to prohibit the sale of sunscreens containing octinoxate and oxybenzone as of Jan. 1, 2021. Safe Suncreen Council; Hogue, 2018. It is likely such environmental-based actions against certain classes of sunscreen components will become more widespread in the future, as the scope of their harm becomes more evident.

### SUMMARY

In some aspects, the presently disclosed subject matter provides a composition comprising one or more crosslinked polysiloxanes having one or more light-filtering compounds covalently or non-covalently bound thereto or otherwise associated therewith.

In certain aspects, the presently disclosed subject matter provides a sunscreen comprising the presently disclosed composition.

In other aspects, the presently disclosed subject matter provides a delivery device comprising the components of the presently disclosed composition.

In yet other aspects, the presently disclosed subject matter provides a method for preparing a composition of claim 1, the method comprising: (a) providing or preparing one or more functionalized organic light-filtering compounds, non-functionalized organic light-filtering compounds, inorganic light-filtering compounds, or combinations thereof; (b) providing or preparing one or more siloxane oligomers; (c) contacting the one or more functionalized organic light-filtering compounds, non-functionalized light-filtering compounds, inorganic light-filtering compounds, or combinations thereof with the one or more siloxane oligomers to form one or more siloxane oligomers labeled with the one or more functionalized UV-filtering compounds or a mixture of the one or more siloxane oligomers with the one or more non-functionalized organic light-filtering compounds, the one or more inorganic light-filtering compounds, or combinations thereof; (d) contacting the one or more siloxane oligomers labeled with the one or more functionalized UV-filtering compounds or a mixture of the one or more siloxane oligomers with the one or more non-functionalized organic light-filtering compounds, the one or more inorganic light-filtering compounds, or combinations thereof with one or more divinylpolysiloxanes, vinylpolysiloxanes, and combinations thereof in the presence of a catalyst to form the presently disclosed composition.

In even yet other aspects, the presently disclosed subject matter provides a “one-pot” method of forming a composition of claim 1, the method comprising: (a) combining one or more functionalized organic light-filtering compounds, non-functionalized organic light-filtering compounds, inorganic light-filtering compounds, or combinations thereof; (b) one or more siloxane oligomers; and (c) one or more divinylpolysiloxanes, vinylpolysiloxanes, and combinations thereof in the presence of a catalyst to form the presently disclosed composition.

In particular aspects, the presently disclosed method comprises forming a film on skin of a subject. In more particular aspects, the film is cured on the skin of the subject.

In other aspects, the presently disclosed subject matter provides a method for attenuating or blocking an amount of radiation from penetrating skin of a subject, the method comprising applying to the skin of the subject at least one of a film or a sunscreen comprising the presently disclosed compositions.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1 shows the absorption ranges of commercial sunscreens (from EltaMD Home Page);

FIG. 2A, FIG. 2B, FIG. 2C, and FIG. 2D show the chemical structures of common organic UV absorbers used in sunscreen (FIG. 2A), including substituted triazine absorbers (FIG. 2B) and miscellaneous UV absorbers (FIG. 2C and FIG. 2D);

FIG. 3A is a reaction scheme illustrating a hydrosilyation reaction and FIG. 3B is a hydrosilylation reaction between multifunctional Si—H and olefins to make crosslinked silicone networks (films). The hydrosilylation reaction is the addition of an Si—H group across an olefin, generally in the presence of a platinum catalyst. These are the reactions used to covalently bond UV-absorbing chromophores to the silicone backbone, as well as to polymerize the precursors into films;

FIG. 4 is a scheme illustrating a two-component approach to skin coatings via hydrosilylation. In this approach, UV absorbers are first functionalized with chemical handles, e.g., an allyl functional group. They are then covalently bound to reactive silicone oligomers, which are subsequently reacted with oligomers bearing complementary functionality (in the presence of a catalyst) to form the crosslinked silicone network (film). The two-component description refers to the minimum number of reactive species participating in the film forming reaction;

FIG. 5 is a scheme illustrating the synthesis of an allyl-functional para-aminobenzoic acid (PABA) derivative;

FIG. 6 is a scheme illustrating the synthesis of an allyl-functional oxybenzone derivative;

FIG. 7 is a scheme illustrating the synthesis of an allyl-functional meradimate. The allyl esters and ethers depicted in FIG. 5, FIG. 6, and FIG. 7 were prepared because they can participate in hydrosilyation reactions and can be readily and efficiently introduced onto functional groups typically present in organic UV-absorbers. Some absorbers are monofunctional leading to exclusive product formation. Other absorbers are multi-functional and can produce multiple reaction products requiring purification;

FIG. 8 is a scheme illustrating the synthesis of meradimate-labeled silicone oligomers and FIG. 9 is a scheme illustrating the synthesis of PABA-labeled silicone oligomers. Chromophore-labeled silicone oligomers, which serve as reactants in the two-component film formation, are first formed by reacting the allyl-functional UV-absorbers with Si—H functional silicone oligomers under hydrosilylation conditions. A key to this reactive step is to use less than a stoichiometric amount of allyl ester (relative to Si—H groups), so as to leave unreacted Si—H groups in the oligomer product to react in the subsequent film-forming step;

FIG. 10 shows ¹H NMR spectra confirming the attachment of the chromophore to the silicone scaffold. The ¹H NMR spectra of the starting reactants (Top, Middle) and the reaction product (Bottom) are shown. The characteristic peaks labeled J in the bottom spectrum shows that the product contains Si—H groups, while peaks labeled E, F, G, and H reveal the presence of the PABA chromophore. Integration of the peaks confirms that the PABA moiety is present in the correct stoichiometry;

FIG. 11 is a scheme illustrating the synthesis of silicone networks via room temperature hydrosilylation;

FIG. 12 is a scheme illustrating the synthesis of PABA-containing silicone polymer networks;

FIG. 13 is a scheme illustrating a one-pot, two-step, three-component approach to functionalized silicone polymers. In this approach, like the previous two-component approach (see FIG. 4), UV absorbers are first functionalized with chemical handles, e.g., allyl functional groups. They are then used directly in the film forming step by combining with Si—H and Si-vinyl functional oligomers (in the presence of a catalyst) to form a crosslinked silicone network (film). The three-component description refers to the minimum number of reactive species participating in the film forming reaction;

FIG. 14 is the structure of allyl cinnamate;

FIG. 15 is a scheme illustrating the synthesis of cinnamate-functional silicones films via a 3-component hydrosilylation approach. Representative variables that were manipulated to identify optimal conditions are presented in Table 2. In this example, conditions used in 042619B worked best. In contrast, high levels of Pt catalyst caused the films to cure too quickly and with many imperfections;

FIG. 16 shows the structures of Q-Resin (idealized) and 1,3-divinyltetramethyldisiloxane. In this example (see also Table 3), silicone film recipes were further refined and found to be improved by the addition of reinforcing resins (e.g., fumed silica and silicone Q-resins) and volatile cure inhibitors. Q-resins were found to be superior to silica for reinforcement (film toughening) because they can be used at lower loading and do not opacify the films. It is possible to combine Q-resins and lower levels of silica. The addition of very low levels of cure inhibitors (e.g., 1,3-divinyltetramethyldisiloxane) was necessary to extend working time and improve film quality (defect and bubble free). These advanced formulations were used to make pristine cinnamate (UVB) labeled films and those containing dispersed (non-covalently) bound ZnO;

FIG. 17 shows photographs of silicone films containing organic and inorganic sunscreen ingredients and FIG. 18 shows UV-vis transmission spectra of (un)labeled silicone films. Labeled and unlabeled silicone films were prepared according to the formulations shown in FIG. 16 and Table 3. Their ability to attenuate incident UV light was measured using a UV-vis spectrometer. Unlabeled silicones blocked very little light, whereas those containing bound cinnamate moieties blocked approximately 100% of the UVB light (below approximately 320 nm), corresponding to the absorption maxima of the cinnamate chromophores. Films containing higher levels of absorber were slightly opaque, due to phase-separated domains within the film—UV blocking was the same. ZnO-containing films blocked nearly all of the UV light—corresponding to the absorption maximum of the pigment. Increasing ZnO concentration is expected to further decrease UV light transmission;

FIG. 19 shows potential structures of allyl-functionalized organic UV filters. These representative compounds are non-limiting examples of other allyl functionalized organic UV-absorbers that can be used to make UV-blocking films. Different absorbers can be combined to cover more of the UV spectrum and potentially affect film service life. Additionally, they can be combined with dispersed inorganic UV filters, such as ZnO or TiO₂, to maximize spectral coverage while minimizing organic content;

FIG. 20 shows representative polysiloxanes suitable for use with the presently disclosed subject matter; and

FIG. 21 shows schematic representations of potential delivery devices for component A and component B of the presently disclosed formulations (left, reactive components A and B are contained in separate bottles or tubes; middle, reactive components A and B are contained in single, dual-chambered tubes or bottles; right, reactive components A and B are contained in a single, dual-chambered syringe-like device.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Functional Skin Coating Polymer

Humans require protection from solar radiation to prevent skin damage and accelerated aging (a process known as photoaging), and to reduce the risks of developing skin cancers. Other than shelter and clothing, humans rely on sunscreens and sunblocks to help reduce the amount of damaging ultraviolet (UV) radiation from penetrating the skin. Sunscreen and sunblock products are typically applied as creams, lotions, gels or sprays. Regardless of the formulation, UV-filtering compounds on the skin surface either absorb or reflect the incoming UV rays. These UV-filtering compounds can be organic molecules or inorganic pigments.

There is a growing body of research indicating that humans also may require protection from visible light, particularly high-energy visible (HEV) light, in the blue-violet region of the spectrum from about 400 nm to about 500 nm. While it does not seem to cause as much direct DNA damage as UV light, blue light has been shown to slow down the rate of skin recovery following damage, and can locally increase pigmentation levels, particularly in people with moderately dark skin, a condition known as melasma, which manifests as a undesirable uneven greyish to brown patches on the skin that can last for long periods of time. This effect is thought to result from synergistic photochemistry that occurs in the presence of UVA light. There is limited data showing that it is possible to protect from visible-light induced pigmentation and improve the fading of melasma by applying products to the skin that contain one or more iron oxides. Like sunscreens, however, these products can be washed from skin, limiting their effectiveness.

To provide the necessary protection from UV or visible light, while reducing the negative effects associated with many sunscreen products, the presently disclosed subject matter provides an alternative paradigm in which the light-filtering compounds are covalently bound to or otherwise closely associated with a polymer film that rests on the skin surface. In this way, the light-filtering compounds can neither be washed nor sweated from the body and cannot diffuse into the skin. These characteristics simultaneously eliminate the need for reapplication and the associated negative health and environmental consequences.

The characteristics of the presently disclosed compositions are especially applicable to infants, babies, toddlers, and the like, as conventional sunscreen formulations for infants, babies, and toddlers have the problems identified and articulated hereinabove. For this vulnerable group, especially with an immature skin barrier function, formulations applied to their skin can get absorbed more easily into the body. Therefore, the presently disclosed compositions, which are specifically designed to eliminate/minimize that possibility, will be a significant safety advancement in sunscreen products for infants, babies, and toddlers.

As used herein, the term “skin” includes the epidermis of a subject's skin, which is the outer layer of the skin and includes the stratified squamous epithelium composed of proliferating basal and differentiated suprabasal keratinocytes. The term skin includes skin associated with any part of the body of a subject. The term “body” includes any part of the subject's body that can benefit from the compositions disclosed herein. Examples of a subject's body include the skin, the neck, the brow, the jowls, the eyes, the hands, the feet, the face, the cheeks, the breasts, the abdomen, the buttocks, the thighs, the back, the legs, the ankles, cellulite, fat deposits, and the like.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

As used herein, the terms “apply,” “applied,” and “application” include any and all known methods of contacting or administering compositions provided herein to a subject's skin or body. The application may be by finger, hand, brush, cotton ball, cotton swab, tissue, pad, sponge, roll-on, spatula, dispenser, drops, spray, splash, foam, mousse, serum, spritz, and other appropriate methods. In particular embodiments, the application includes a spray application.

In some embodiments, the presently disclosed subject matter provides a family of film-forming polymers that contain covalently bound or non-covalently associated light-filtering compounds. The presently disclosed approach is general and can work with nearly any appropriately-functionalized organic light-absorbing chromophore. This approach was demonstrated by first pre-attaching the organic filters to an oligomeric silicone scaffold and second, in which all components are reacted during the film forming step (without preforming the chromophore-labeled intermediate). More particularly, in representative embodiments, it has been shown that silicone films containing various levels of a model organic UV absorber, e.g., cinnamate, can cure into flexible, elastomeric films having sufficiently high tear strength to be useful as a skin protectant.

Equally important, the presently disclosed chromophore labeled films completely block UV transmission through the film at wavelengths corresponding to the absorption maxima of the UV-absorber and have been shown to exhibit concentration-dependent absorption properties. Further, it has been shown that silicone films containing dispersed, non-covalently associated, inorganic UV reflecting pigments (e.g., ZnO) also are effective at reducing the transmission of UV light over a broader range of the spectrum. Accordingly, inorganic pigments, such as TiO₂, ZnO, zirconium oxide, cerium oxide, one or more iron oxides, and combinations thereof, can be used independently and/or in combination with organic UV filters as an approach to broad spectrum UV filtering.

Conventional sunscreens consist of dispersions of UV-filtering compounds in media of varying volatility. Upon application to the skin, the medium evaporates, leaving behind a layer of non-connected, unbound UV-filtering compounds that can be washed, perspired, or rubbed off. This drawback leads to potential absorption of the UV-filtering compounds by the body and/or release into the environment. In addition, this conventional approach requires periodic re-application of the sunscreen to maintain protection.

The presently disclosed subject matter further differs from sunscreen technology known in the art in that the media in which the light-filtering compounds are dispersed is not volatile and is a reactive, film forming, polymer matrix. Films are formed directly on the skin surface by chemical reaction (polymerization) of its constituents. In one embodiment, the light-filtering compounds can be covalently bound to the film. In another embodiment, the light-filtering compounds are not covalently bound, but are closely associated to the polymer through favorable non-covalent interactions including, but not limited to, hydrogen bonds, van der Waals interactions, π-stacking, hydrophobic interactions, or combinations thereof. By virtue of the covalent bonding or favorable non-covalent associations, the light-filtering compounds are trapped within the film and cannot be substantially washed away, or otherwise released into the skin or into the environment. The films, which preferentially are derived from breathable polymeric materials, will stay in place, continuing to block radiation until they are intentionally removed by the applicant. Once formulated, these light-blocking skin coatings can be used to replace existing conventional sunscreen lotions. Moreover, since the light-filtering compounds used are covalently attached to and/or closely associated with the polymer and cannot be washed from the films, they can employ well established light-filtering compounds, including those that perform well, but have been discontinued or outlawed due to unfavorable health or environment effects.

Accordingly, in some embodiments, the presently disclosed subject matter provides a composition comprising one or more crosslinked polysiloxanes having one or more light-filtering compounds covalently or non-covalently bound thereto or otherwise associated therewith.

In certain embodiments, the one or more crosslinked polysiloxanes are prepared by reacting at least one Si—H functional polymer/oligomer with at least one Si-vinyl functional polymer/oligomer, or combinations thereof. In representative embodiments, the at least one Si—H functional polymer/oligomer and least one Si-vinyl functional polymer/oligomer are selected from the group consisting of polymethylhydrosiloxane, polymethylhydrosiloxane copolymers, divinylpolysiloxane, vinylpolysiloxane, monovinyl, monohydride terminated polysiloxane, and combinations thereof. In particular embodiments, the at least one Si—H functional polymer/oligomer is selected from compounds of formulae (I)-(IV) provided immediately herein below. In particular embodiments, the at least one Si-vinyl functional polymer/oligomer is selected from compound of formulae (V)-(IX) provided herein below. One of ordinary skill in the art would recognize that other Si—H functional polymers/oligomers and Si-vinyl functional polymers/oligomers would be suitable for use with the presently disclosed subject matter.

In particular embodiments, the one or more crosslinked polysiloxanes comprise one or more polymethylhydrosiloxanes selected from the group consisting of:

(a)

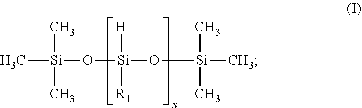

wherein:

R₁ is selected from the group consisting of hydrogen, C₁-C₂₀ alkyl, C₁-C₂₀ cycloalkyl, C₁-C₂₀ unsaturated alkyl, C₁-C₂₀ haloalkyl, hydroxylalkyl, alkoxyalkyl, carboxyalkyl, C₆-C₂₀ aryl, substituted aryl; and

x is an integer from 2 to 500;

(b)

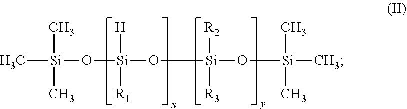

wherein:

R₁, R₂, R₃ are the same or different and are each selected from the group consisting of C₁-C₂₀ alkyl, C₁-C₂₀ cycloalkyl, C₁-C₂₀ unsaturated alkyl, C₁-C₂₀ haloalkyl, hydroxylalkyl, alkoxyalkyl, carboxyalkyl, C₆-C₂₀ aryl, and substituted aryl; and

x and y are each independently an integer from 1 to 500;

(c)

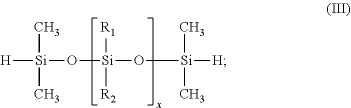

wherein:

R₁ and R₂ are the same or different and are each selected from the group consisting of hydrogen, C₁-C₂₀ alkyl, C₁-C₂₀ cycloalkyl, C₁-C₂₀ unsaturated alkyl, C₁-C₂₀ haloalkyl, hydroxylalkyl, alkoxyalkyl, carboxyalkyl, C₆-C₂₀ aryl, and substituted aryl; and

x is an integer from 1 to 1000; and

(d)

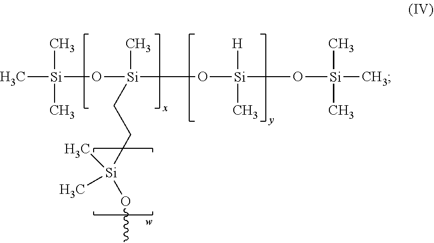

R₁ and R₂ are the same or different and are each selected from the group consisting of hydrogen, C₁-C₂₀ alkyl, C₁-C₂₀ cycloalkyl, C₁-C₂₀ unsaturated alkyl, C₁-C₂₀ haloalkyl, hydroxylalkyl, alkoxyalkyl, carboxyalkyl, C₆-C₂₀ aryl, and substituted aryl; and

x, y, and w are each independently an integer from 1 to 1000.

In particular embodiments, the one or more polysiloxanes comprise one or more divinylpolysiloxanes or vinylpolysiloxanes selected from the group consisting of:

(a)

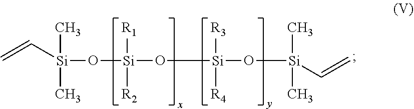

wherein:

R₁, R₂, R₃ and R₄ are the same or different and are selected from the group consisting of C₁-C₂₀ alkyl, C₁-C₂₀ cycloalkyl, C₁-C₂₀ unsaturated alkyl, C₁-C₂₀ haloalkyl, hydroxylalkyl, alkoxyalkyl, carboxyalkyl, C₆-C₂₀ aryl, and substituted aryl; and

x and y are each independently an integer from 1 to 500;

(b)

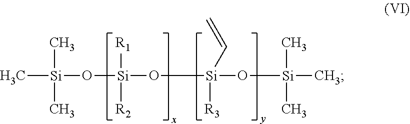

wherein:

R₁, R₂, and R₃ are the same or different and are each selected from the group consisting of C₁-C₂₀ alkyl, C₁-C₂₀ cycloalkyl, C₁-C₂₀ unsaturated alkyl, C₁-C₂₀ haloalkyl, hydroxylalkyl, alkoxyalkyl, carboxyalkyl, C₆-C₂₀ aryl, and substituted aryl; and

x is an integer from 0 to 500; and

y is an integer from 2 to 1000;

(c)

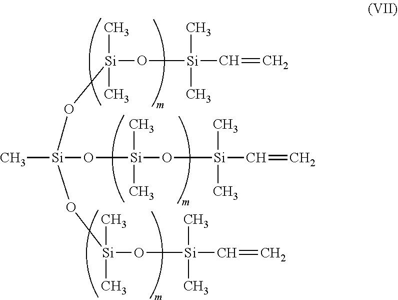

wherein each m is independently an integer from 1 to 100;

(d)

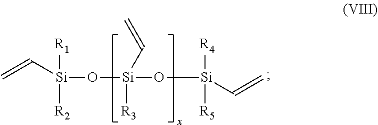

wherein;

R₁, R₂, R₃, R₄, and R₅ are the same or different and are each selected from the group consisting of C₁-C₂₀ alkoxyl, C₁-C₂₀ alkyl, C₁-C₂₀ cycloalkyl, C₁-C₂₀ unsaturated alkyl, C₁-C₂₀ haloalkyl, hydroxylalkyl, alkoxyalkyl, carboxyalkyl, C₆-C₂₀ aryl, and substituted aryl; and

y is an integer from 1 to 1000; and

(e)

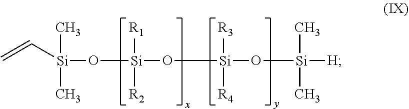

wherein:

R₁, R₂, R₃ and R₄ are the same or different and are selected from the group consisting of C₁-C₂₀ alkyl, C₁-C₂₀ cycloalkyl, C₁-C₂₀ unsaturated alkyl, C₁-C₂₀ haloalkyl, hydroxylalkyl, alkoxyalkyl, carboxyalkyl, C₆-C₂₀ aryl, and substituted aryl; and

x and y are each independently an integer from 1 to 500.

In certain embodiments, the one or more polysiloxanes comprise a crosslinked polysiloxane network.

In certain embodiments, the one or more crosslinked polysiloxanes form a covalently bound adduct with the one or more light-filtering compounds. In some embodiments, the adduct has the following general formula:

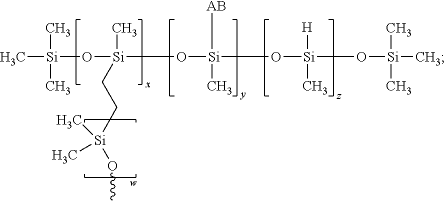

wherein AB is a light-filtering compound; and x, y, and z are each independently an integer from 1 to 1000.

In illustrative embodiments, a crosslinked polysiloxane can form an adduct with PABA:

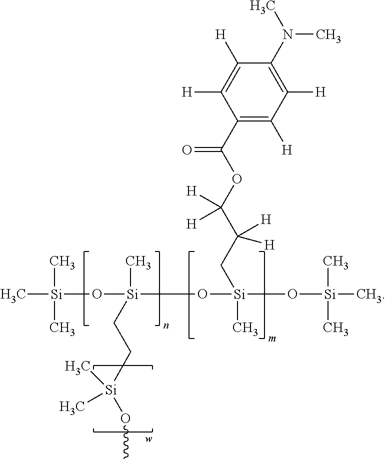

In some embodiments, the one or more light-filtering compounds comprise one or more organic light-filtering compounds. As used herein, the term “light-filtering compound” and derivatives thereof includes compounds that absorb light in the UV-spectral region, e.g., from about 315 nm to about 400 nm (UVA); from about 280 nm to about 315 nm (UVB), and from about 180 nm to about 280 nm (UVC); the visible-spectral region, e.g., from about 400 nm to about 700 nm; and the near-infrared spectral region, e.g., from about 750 nm to about 2,500 nm. The light-filtering compound can absorb radiation, reflect radiation, or scatter radiation such that the radiation does not penetrate or pass through the protective coating.

In particular embodiments, the one or more organic light-filtering compounds comprise one or more organic UV-filtering compounds. In more particular embodiments, the one or more organic UV-filtering compounds is selected from the group consisting of:

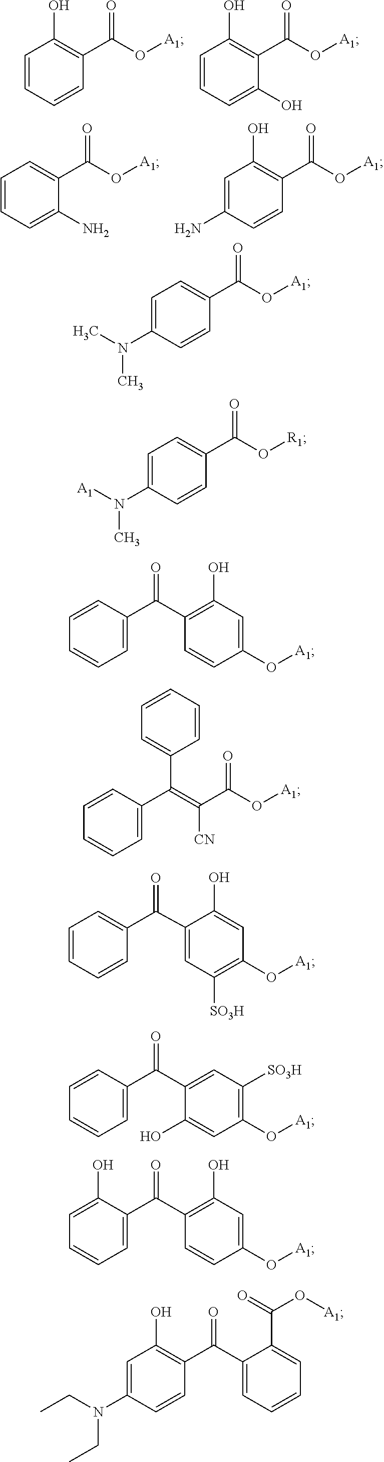

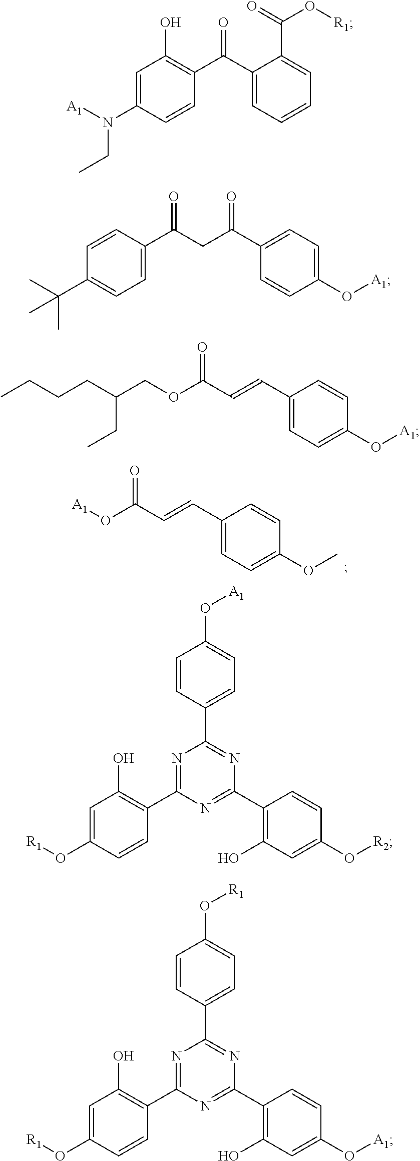

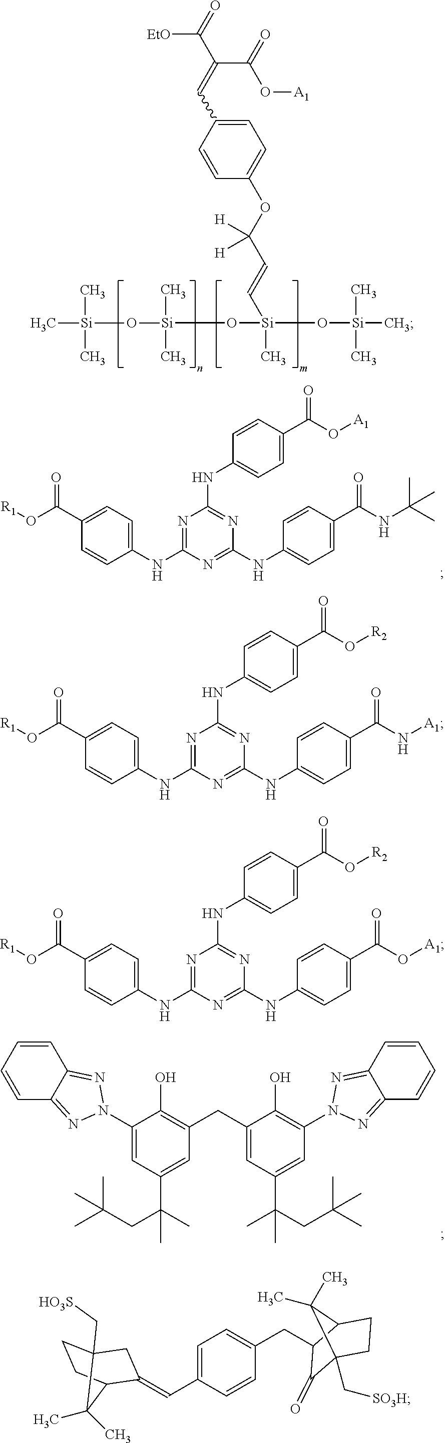

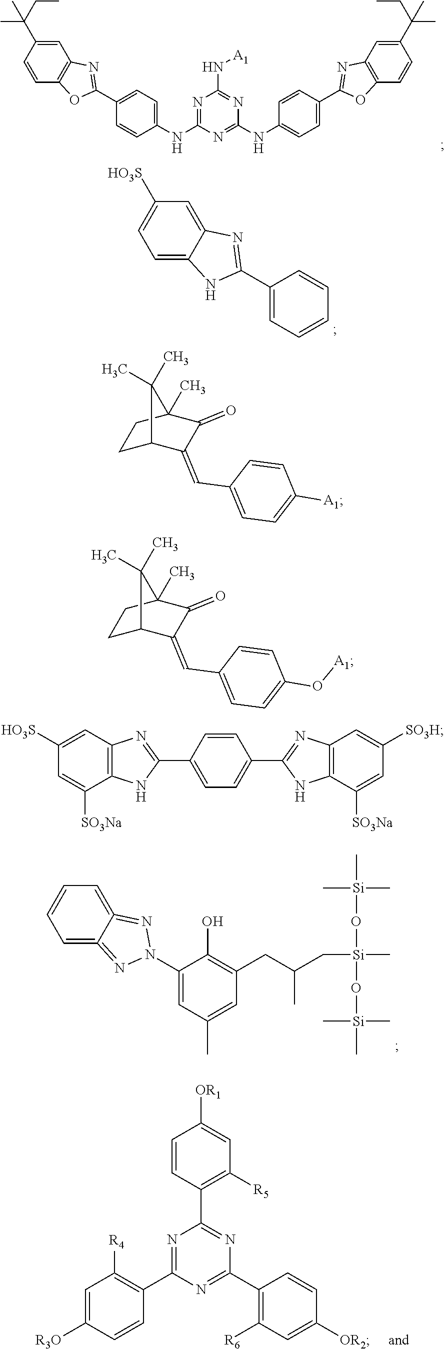

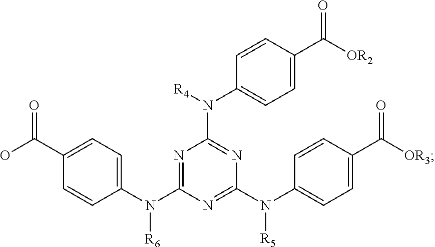

wherein:
A₁ is a moiety comprising an unsaturated hydrocarbon that can undergo hydrosilylation or A₁ can comprise one or more of R₁, R₂, R₃, R₄, and R₅ as defined immediately herein below;
R₁, R₂, and R₃ are each independently selected from the group consisting of H, C₁-C₂₀ alkyl, aryl, and cycloalkyl; and
R₄, R₅, and R₆ are each independently selected from the group consisting of H, hydroxyl, C₁-C₂₀ alkoxyl, aryloxyl, and cycloalkoxyl.

In particular embodiments, A₁ comprises at least one alkenyl or alkynyl group capable of undergoing hydrosilylation and covalent attachment to the crosslinked polysiloxane at the Si—H positions via hydrosilylation. One of ordinary skill in the art would recognize that the one or more organic UV-filtering compounds having an A₁ moiety comprising an unsaturated hydrocarbon that can undergo hydrosilylation is capable of covalently bonding with the one or more crosslinked polysiloxanes. In other embodiments, the organic UV-filtering compounds lacking an A₁ moiety, or embodiments of organic UV-filtering compounds in which A₁ comprises one or more of R₁, R₂, R₃, R₄, and R₅, are capable of being non-covalently associated with the one or more crosslinked polysiloxanes, e.g., through hydrogen bonding, a van der Waals interaction, π-stacking, a hydrophobic interaction, or combinations thereof. In some embodiments, the presently disclosed compositions comprise a combination or blend of one or more covalently bound organic UV-filtering compounds and one or more non-covalently bound UV-filtering compounds.

In some embodiments, the one or more organic UV-filtering compounds is selected from the group consisting of

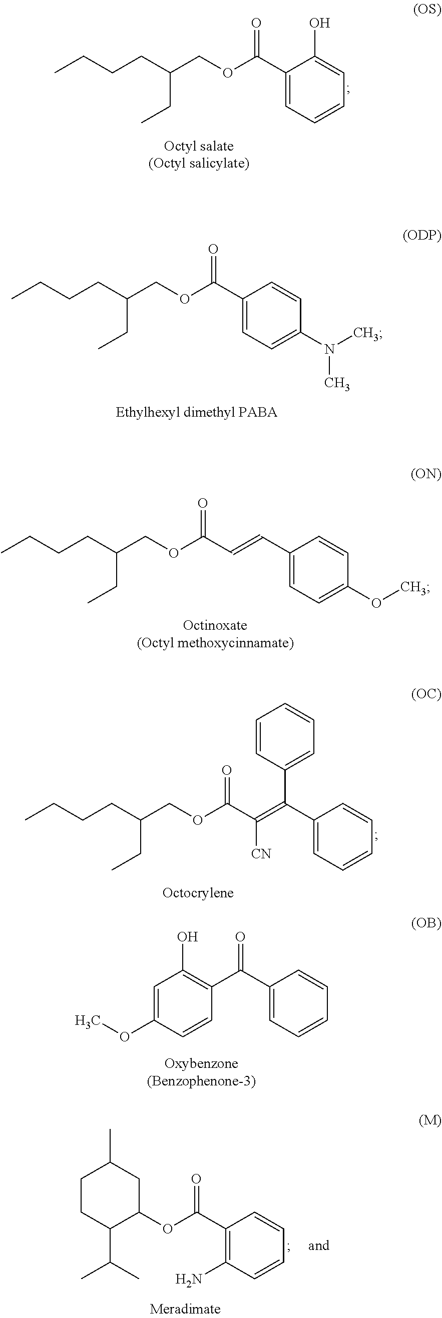 [SMILES: CCCCC(CC)COC(=O)c1ccccc1O ; CCCCC(CC)COC(=O)c1ccc(N(C)C)cc1 ; CCCCC(CC)COC(=O)/C=C/c1ccc(OC)cc1 ; CCCCC(CC)COC(=O)C(C#N)=C(c1ccccc1)c1ccccc1 ; COc1ccc(C(=O)c2ccccc2)c(O)c1 ; CC1CCC(C(C)C)C(OC(=O)c2ccccc2N)C1]

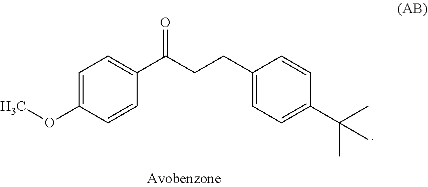 [SMILES: COc1ccc(C(=O)CCc2ccc(C(C)(C)C)cc2)cc1]

In other embodiments, the one or more organic UV-filtering compounds comprises a reactive UV-filtering compound selected from the group consisting of

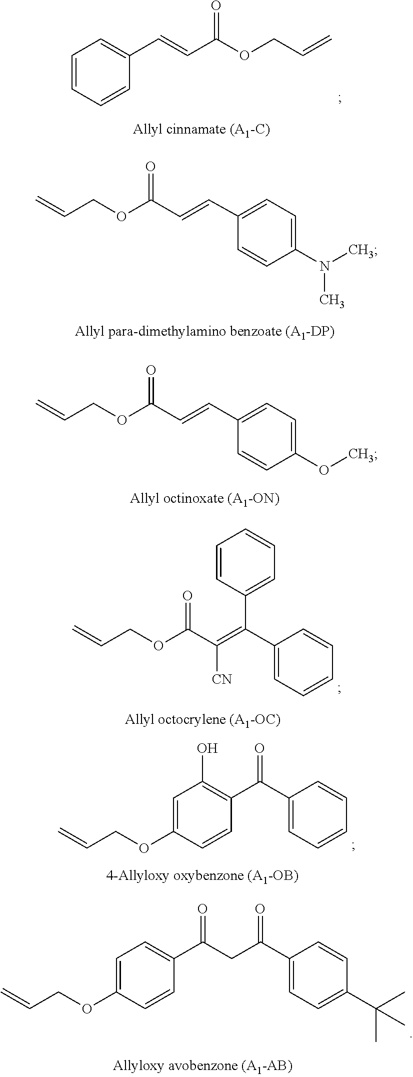

In other embodiments, the light-filtering compound comprises an inorganic light-filtering compound. In particular embodiments, the inorganic light-filtering compound is selected from the group consisting of zinc oxide, titanium oxide, iron oxide, zirconium oxide, cerium oxide, and combinations thereof.

In yet other embodiments, the presently disclosed composition further comprises a Q-resin. In certain embodiments, the Q-resin is selected from the group consisting of:

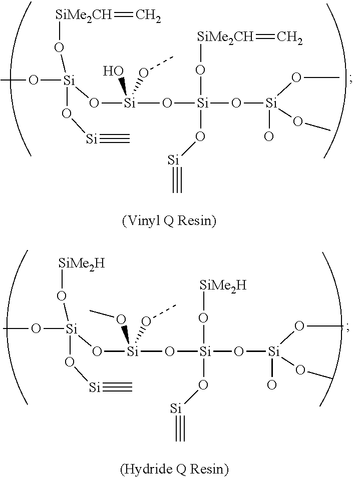

and combinations thereof.

In other embodiments, the presently disclosed composition further comprises an organic or inorganic reinforcing filler. In certain embodiments, the inorganic reinforcing filler is selected from the group consisting of a clay, chalk, talc, calcite (CaCO₃), mica, barium sulfate, zirconium dioxide, zinc sulfide, zinc oxide, titanium dioxide, aluminum oxide, silica aluminates, calcium silicates, and a surface-treated silica. In particular embodiments, the reinforcing filler is selected from the group consisting of Al₂O₃ and SiO₂. In certain embodiments, the surface-treated silica is selected from the group consisting of fumed silica, hydrated silica, and anhydrous silica.

In some embodiments, the one or more non-covalently bound light-filtering compounds are associated with the one or more crosslinked polysiloxanes by one or more of a hydrogen bond, a van der Waals interaction, π-stacking, a hydrophobic interaction, or combinations thereof.

In certain embodiments, the presently disclosed subject matter provides a sunscreen comprising the presently disclosed composition.

In yet other embodiments, the presently disclosed subject matter provides a delivery device comprising the presently disclosed composition or components thereof. In particular embodiments, the delivery device comprises two or more reactive components of the presently disclosed composition, wherein the two or more reactive components are contained in one or more of:

(a) separate bottles or tubes;

(b) a single, dual-chambered tube or bottle comprising a first chamber and a second chamber; and

(c) a single, dual-chambered syringe-like device comprising a first chamber and a second chamber.

A representative dual-chambered syringe is disclosed in International PCT Patent Application Publication No. WO2018052951A1 to Shienlin for Kits, Compositions and Methods for Wound Treatment and Management, published Mar. 22, 2018, which is incorporated by reference in its entirety.

In some embodiments, the first chamber of the dual-chambered tube, bottle or syringe-like device comprises:

(a) a mixture of one or more polymethylhydrosiloxanes, one or more divinyl- or vinylpolysiloxanes, and one or more light-filtering compounds, whereas the second chamber comprises a catalyst and optionally one or more inhibitors; or

(b) a mixture of one or more polymethylhydrosiloxanes, one or more divinyl- or vinylpolysiloxanes, and one or more light-filtering compounds, whereas the second chamber comprises one or more divinyl- or vinylpolysiloxanes, a catalyst and optionally one or more inhibitors.

In some embodiments, the catalyst comprises an emulsion, as described in International PCT Patent Application Publication No. WO2020067582A1 to Akthakul et al., for Compositions and methods for application over skin, published Apr. 2, 2020, which is incorporated by reference in its entirety.

In other embodiments, the presently disclosed subject matter provides a method for preparing the presently disclosed composition, the method comprising:
(a) providing or preparing one or more functionalized organic light-filtering compounds, non-functionalized organic light-filtering compounds, inorganic light-filtering compounds, or combinations thereof;
(b) providing or preparing one or more siloxane oligomers;
(c) contacting the one or more functionalized organic light-filtering compounds, non-functionalized light-filtering compounds, inorganic light-filtering compounds, or combinations thereof with the one or more siloxane oligomers to form one or more siloxane oligomers labeled with the one or more functionalized UV-filtering compounds or a mixture of the one or more siloxane oligomers with the one or more non-functionalized organic light-filtering compounds, the one or more inorganic light-filtering compounds, or combinations thereof;
(d) contacting the one or more siloxane oligomers labeled with the one or more functionalized UV-filtering compounds or a mixture of the one or more siloxane oligomers with the one or more non-functionalized organic light-filtering compounds, the one or more inorganic light-filtering compounds, or combinations thereof with one or more divinylpolysiloxanes, vinylpolysiloxanes, and combinations thereof in the presence of a catalyst to form the presently disclosed composition.

In certain embodiments, the one or more siloxane oligomers comprise one or more polymethylhydrosiloxanes provided hereinabove.

In certain embodiments, the one or more divinylpolysiloxanes or vinylpolysiloxanes comprise the one or more divinylpolysiloxanes or vinylpolysiloxanes provided hereinabove.

In particular embodiments, the presently disclosed method further comprises adding a Q-resin to the composition. In more particular embodiments, the Q-resin is a Q-resin as provided hereinabove.

In some embodiments, the catalyst comprises a hydrosilylation catalyst. In certain embodiments, the hydrosilylation catalyst comprises a metal. In particular embodiments, the metal is selected from the group consisting of platinum, rhodium, tin, or a combination thereof. In more particular embodiments, the metal is platinum and the hydrosilylation catalyst is selected from the group consisting of a platinum carbonyl cyclovinylmethylsiloxane complex, a platinum divinyltetramethyldisiloxane complex, a platinum cyclovinylmethylsiloxane complex, a platinum octanaldehyde/octanol complex, and combinations thereof.

In other embodiments, the metal is rhodium and the hydrosilylation catalyst is tris(dibutyl sulfide) rhodium trichloride.

In yet other embodiments, the metal is tin and the hydrosilylation catalyst is selected from the group consisting of tin II octanoate, tin II neodecanoate, dibutyltin diisooctylmaleate, di-n-butyl bis-(2,4pentanedionate)tin, di-n-butylbutoxychlorotin, dibutyltin dilaurate, dimethyltin dineodecanoate, dimethylhydroxy(oleate) tin, tin II oleate, and a combinations thereof.

In some embodiments, the presently disclosed method further comprises adding an organic or inorganic reinforcing filler to the composition. In certain embodiments, the inorganic reinforcing filler is selected from the group consisting of a clay, chalk, talc, calcite (CaCO₃), mica, barium sulfate, zirconium dioxide, zinc sulfide, zinc oxide, titanium dioxide, aluminum oxide, silica aluminates, calcium silicates, and a surface-treated silica.

In certain embodiments, the reinforcing filler is selected from the group consisting of Al₂O₃ and SiO₂. In certain embodiments, the surface-treated silica is selected from the group consisting of fumed silica, hydrated silica, and anhydrous silica.

In other embodiments, the presently disclosed subject matter provides a “one-pot” method of forming the presently disclosed composition, the method comprising:
(a) combining one or more functionalized organic light-filtering compounds, non-functionalized organic light-filtering compounds, inorganic light-filtering compounds, or combinations thereof;
(b) one or more siloxane oligomers; and
(c) one or more divinylpolysiloxanes, vinylpolysiloxanes, and combinations thereof in the presence of a catalyst to form a composition of claim 1.

In certain embodiments, the one or more siloxane oligomers comprise one or more polymethylhydrosiloxanes as provided hereinabove.

In certain embodiments, the one or more divinylpolysiloxanes or vinylpolysiloxanes comprise the one or more divinylpolysiloxanes or vinylpolysiloxanes as provided hereinabove.

In other embodiments, the presently disclosed method further comprises adding a Q-resin to the composition. In particular embodiments, the Q-resin is a Q-resin as provided hereinabove.

In certain embodiments, the catalyst comprises a hydrosilylation catalyst. In particular embodiments, the hydrosilylation catalyst comprises a metal. In more particular embodiments, the metal is selected from the group consisting of platinum, rhodium, tin, or a combination thereof. In yet more particular embodiments, the metal is platinum and the hydrosilylation catalyst is selected from the group consisting of a platinum carbonyl cyclovinylmethylsiloxane complex, a platinum divinyltetramethyldisiloxane complex, a platinum cyclovinylmethylsiloxane complex, a platinum octanaldehyde/octanol complex, and combinations thereof. In even yet more particular embodiments, the catalyst is Karstedt's catalyst:

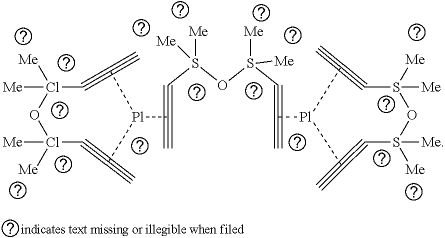

In other embodiments, the metal is rhodium and the hydrosilylation catalyst is tris(dibutyl sulfide) rhodium trichloride. In yet other embodiments, the metal is tin and the hydrosilylation catalyst is selected from the group consisting of tin II octanoate, tin II neodecanoate, dibutyltin diisooctylmaleate, di-n-butyl bis-(2,4pentanedionate)tin, di-n-butylbutoxychlorotin, dibutyltin dilaurate, dimethyltin dineodecanoate, dimethylhydroxy(oleate) tin, tin II oleate, and a combinations thereof.

In some embodiments, the method further comprises adding an organic or inorganic reinforcing filler to the composition. In certain embodiments, the inorganic reinforcing filler is selected from the group consisting of a clay, chalk, talc, calcite (CaCO₃), mica, barium sulfate, zirconium dioxide, zinc sulfide, zinc oxide, titanium dioxide, aluminum oxide, silica aluminates, calcium silicates, and a surface-treated silica. In particular embodiments, the reinforcing filler is selected from the group consisting of Al₂O₃ and SiO₂. In certain embodiments, the surface-treated silica is selected from the group consisting of fumed silica, hydrated silica, and anhydrous silica.

In other embodiments, the presently disclosed method further comprises forming a film comprising the presently disclosed composition. In particular embodiments, the method further comprises forming a film on skin of a subject. In more particular embodiments, the film is cured on the skin of the subject.

In other embodiments, the presently disclosed subject matter provides a method for attenuating or blocking an amount of radiation from penetrating skin of a subject, the method comprising applying to the skin of the subject at least one of a film or a sunscreen comprising the presently disclosed composition.

### II. Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁₋₁₀ means one to ten carbons, including 1, 2, 3, 4, 5, 6, 7, 8, 9, and 10 carbons). In particular embodiments, the term “alkyl” refers to C₁-20 inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, 2-ethylhexyl, isodecyl, and homologs and isomers thereof.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, cyano, and mercapto.

“Cyclic” and “cycloalkyl” refer to a non-aromatic mono- or multicyclic ring system of about 3 to about 20 carbon atoms, e.g., 3, 4, 5, 6, 7, 8, 9, 10, 11, 12, 13, 14, 15, 16, 17, 18, 19, and 20 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

As used herein, an “alkoxy” group is an alkyl attached to the remainder of the molecule through a divalent oxygen. The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

The term “carboxyalkyl” includes a radical or group comprising an alkyl and a carboxy group. A carboxy group refers to the —COOH group.

The term “aryl” means, unless otherwise stated, an aromatic hydrocarbon substituent that can be a single ring or multiple rings (such as from 1 to 3 rings), which are fused together or linked covalently.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments±50%, in some embodiments ±20%, in some embodiments ±10%, in some embodiments ±5%, in some embodiments±1%, in some embodiments±0.5%, and in some embodiments±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Functional Skin Coating Polymer

### 1.1 Overview

Recognizing the broad and continued need for improved and sustained UV protection, together with the simultaneous responsibility to address the biological and ecological consequences presented by conventional sunscreen components, the presently disclosed subject matter provides an improved approach for human sun protection. The presently disclosed subject matter, comprises light-filtering compounds covalently bound or otherwise closely associated with a polymer film positioned directly on the skin. This approach would prevent the light-filtering compounds from being washed or sweated off, and prevent them from leaching into the skin or the environment.

More particularly, the presently disclosed subject matter provides a novel, polymer film-based coatings platform that can be easily applied to and removed from the skin. The polymer film-based coating provides tailored, long-lasting protection from a variety of external agents. Such durable skin coatings could conceivably provide extended protection from harmful UV and other solar radiation.

### 1.2 Technical Approach

To achieve the above-stated goal of binding or associated light-filtering compounds within a polymeric film on the skin, the film-forming polymer that would serve as the host matrix for those components was considered. Silicones were identified as being a suitable polymer. As used herein, a silicone (also referred to herein as a “polysiloxane”) is a polymer made up of repeating units of siloxane, i.e., a chain of alternating silicon atoms and oxygen atoms, combined with carbon, hydrogen, and sometimes other elements. Most importantly, silicone polymers are inherently biocompatible, hypoallergenic, and poorly absorbed through the skin. Many silicone derivatives and precursors have been approval by the U.S. Food and Drug Administration (FDA) for skin contact applications. See Klykken et al.

For those reasons, silicones have been utilized for a variety of medical and cosmetic purposes. In the cosmetics industry, silicones are valued for their softness, as well as for their ability to form tough elastomers for film and mask applications, for which they are particularly well suited due to their ability to achieve skin-like feel and flexibility, as well as exceptional breathability. Interestingly, despite their high O₂ and water vapor permeability, silicones are exceptionally hydrophobic materials and serve as excellent barriers to liquid water. Therefore, silicones are often used as the basis for water-resistant coatings.

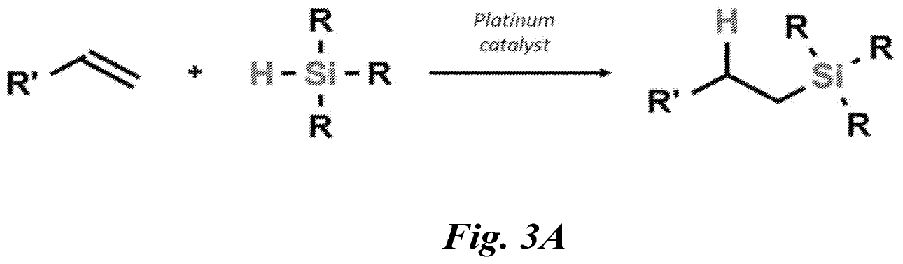

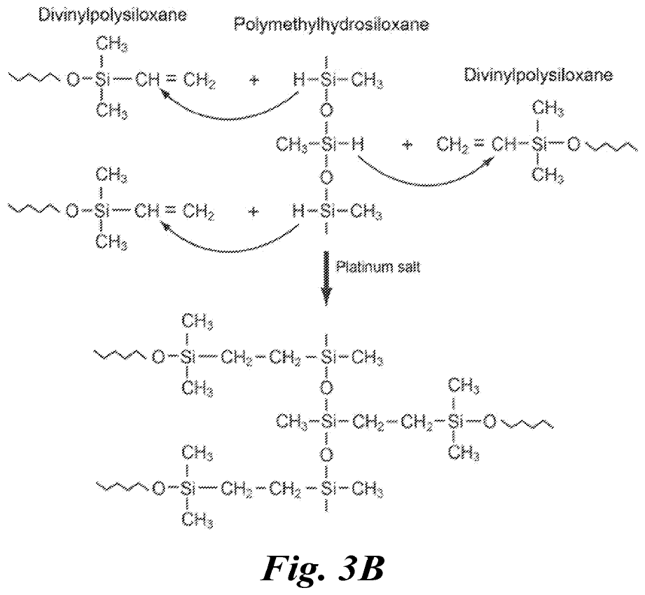

From a chemical perspective, silicone polymers (and their networks) can be formed via one of two very reliable and high-yielding chemical transformations: (1) the condensation of chlorosilanes or alkoxysilanes or (2) hydrosilylation. The hydrosilylation route involves the transition metal (typically platinum) catalyzed addition of a hydrosilane (—Si—H) across a carbon-carbon double bond, typically a terminal olefin (i.e., —CH═CH₂), and can be used for both small molecule manipulations and polymerization (FIG. 3). Hofmann et al., 2017.

Depending on the specific catalyst, the reaction is generally fast at room temperature, extremely tolerant of other functional groups, and efficient with respect to the catalyst demand. The hydrosilylation approach has been used as the basis for specialty skin tightening (anti-wrinkle) coatings in cosmetic applications. U.S. Pat. No. 8,691,202 to Yu et al., for Skin Compositions and Methods of Use Thereof, issued Apr. 8, 2014, which is incorporated herein by reference in its entirety. See also:

U.S. Pat. No. 9,114,096 to Yu et al., for Skin Compositions and Methods of Use Thereof, issued Aug. 25, 2015;

U.S. Pat. No. 9,308,221 to Yu et al., for Skin Compositions and Methods of Use Thereof, issued Apr. 12, 2016;

U.S. Pat. No. 9,333,223 to Yu et al., for Compositions and Methods for Treating Conditions of Compromised Skin Barrier Function, issued May 10, 2016;

U.S. Pat. No. 9,511,034 to Garrett, for Method for Applying a Skin Treatment, issued Dec. 6, 2016;

International PCT Patent Application Publication No. WO2018052951A1 to Shienlin for Kits, Compositions and Methods for Wound Treatment and Management, published Mar. 22, 2018;

U.S. Pat. No. 10,543,161 to Farran et al., for Methods for Protecting and Improving the Appearance of Skin, issued Jan. 28, 2020; and

International PCT Patent Application Publication No. WO2020067582A1 to Akthakul et al., for Compositions and methods for application over skin, published Apr. 2, 2020, each of which is incorporated by reference in its entirety.

When both the Si—H and Si-olefin reactants (monomers) bear exactly two reactive groups, linear polymers are produced. When one or both monomers possess three or more reactive moieties, however, 3-dimensional, crosslinked polymeric networks are formed, with the thinnest form being a thin film.

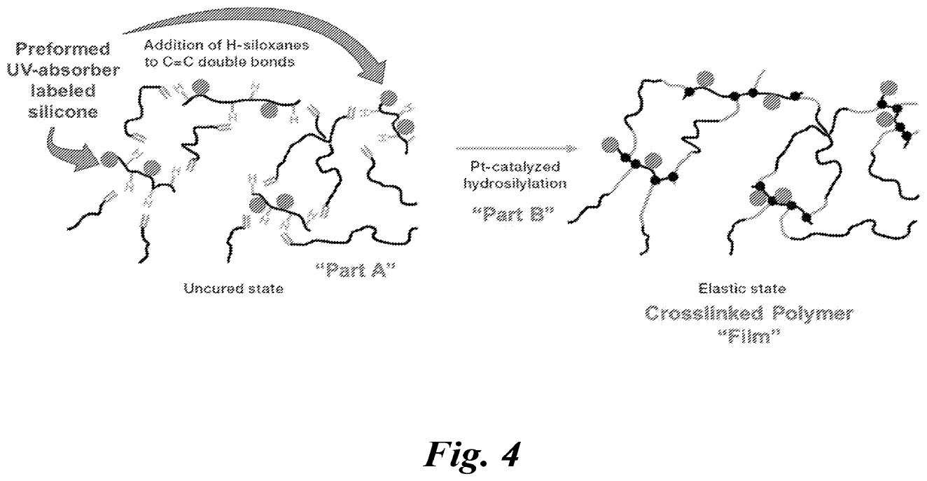

Based on this understanding, development of a UV-absorbing silicone-based skin-coating platform by copolymerizing silicone monomers that were previously derivatized with covalently-attached UV-absorbing chromophores was envisioned (FIG. 4). Both the extent and wavelengths of absorption could be tailored by the specific choice and loading of the functionalized monomers. Monomers labeled with different UV absorbers could be combined in the copolymerization step to yield polymer films having broad-spectrum absorption. Both functionalized and unlabeled silicone precursors can be formulated into a 2-part product that rapidly cures into a tough, robust, yet flexible and breathable silicone film directly on the skin using Karstedt's catalysts, an FDA-approved platinum catalyst complex.

Covalently binding the chromophores to the polymeric scaffold prevents their loss by leaching, evaporation, or diffusion into the skin, which in addition to providing longer lasting performance, should prove ultimately less harmful for both the consumer and the environment. The functional films will remain in place on the skin until they are peeled away and discarded.

The presently disclosed approach to developing UV-absorbing silicone-based skin coatings materials includes the following aspects: (1) derivatize UV-absorbing chromophores with olefinic functional groups; (2) conjugate the functionalized chromophore to oligomeric silicone scaffolds; (3) copolymerize the chromophore-labeled scaffolds into films for characterization; and (4) formulate the chromophore-labeled scaffolds into two-part recipes for direct skin application.

1.2.1 Derivatization of UV-Absorbing Chromophores with Olefinic Functional Groups

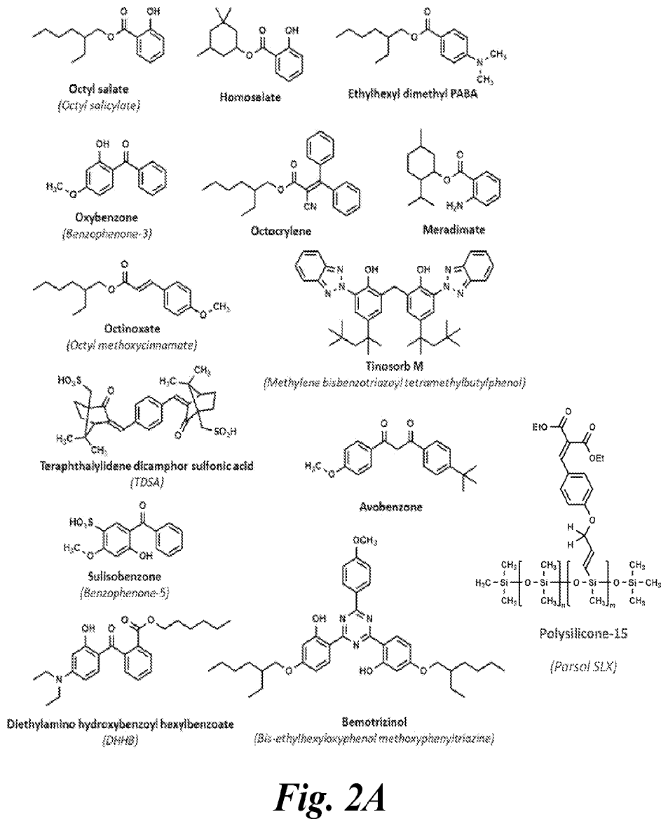

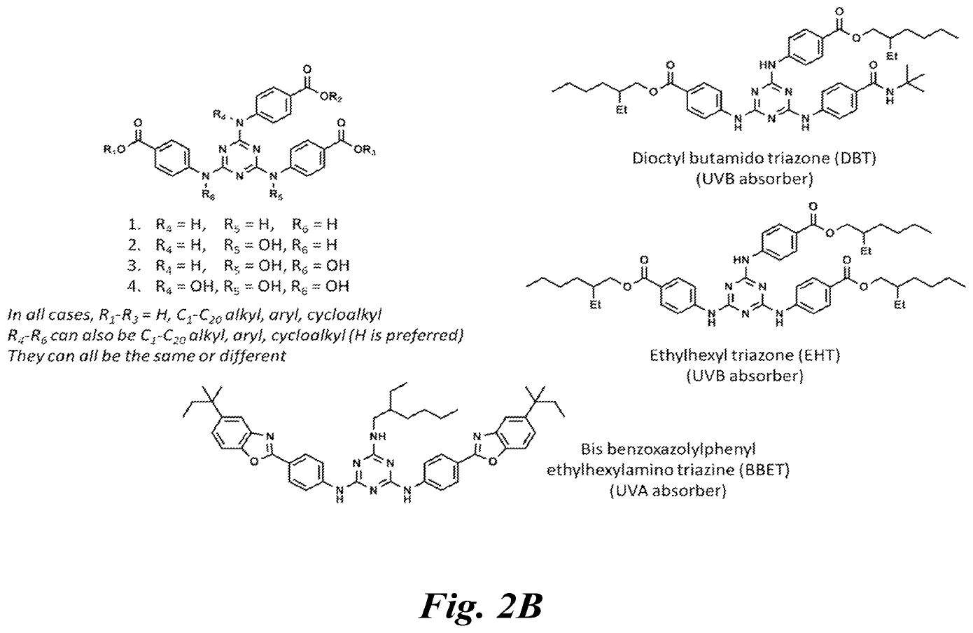

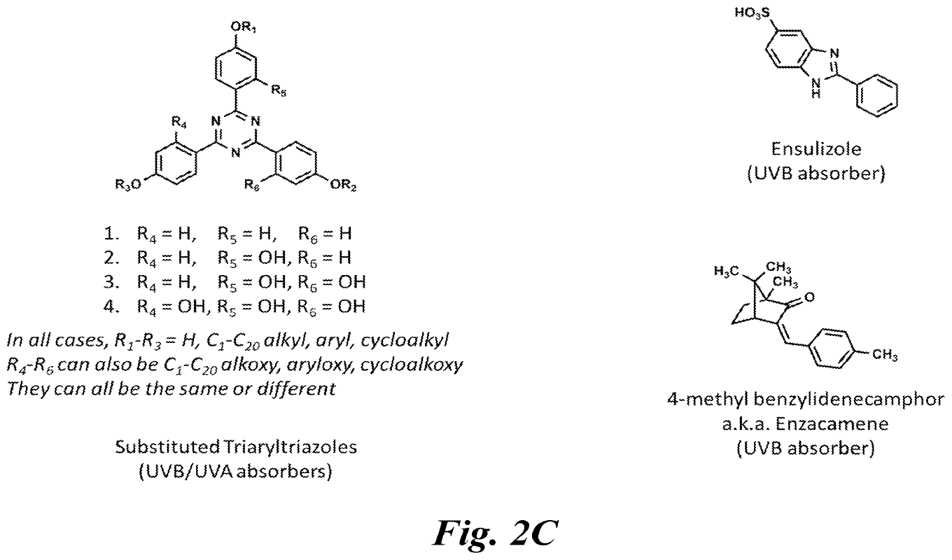

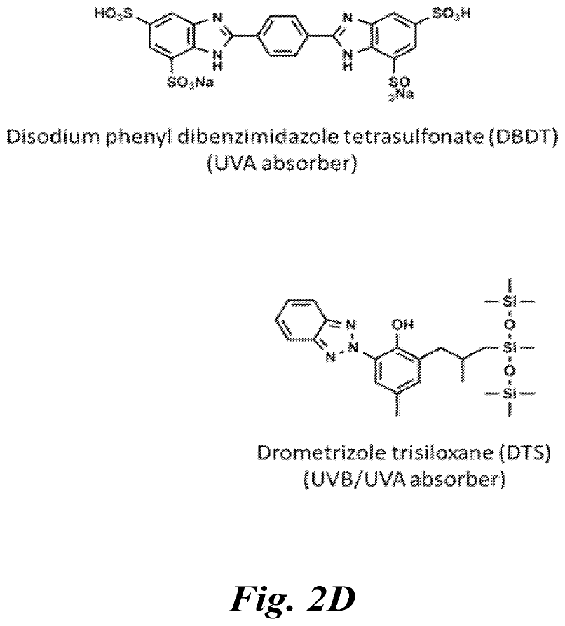

The approach to this task included derivatizing UV-absorbing chromophores with allylic (—CH₂CH═CH₂) functional groups that would serve as the chemical handles for subsequent reactions. Allyl groups were chosen because they exhibit good reactivity in hydrosilylation reactions and because they can be readily installed on the types of nucleophilic functional groups present (e.g., carboxyates, phenols, and amines) on many common UV-absorbing chromophores (see FIG. 2). One of ordinary skill in the art would recognize that other alkenyl or alkynyl groups groups are suitable for use with the presently disclosed subject matter.

As used herein, the term “alkenyl” refers to a monovalent group derived from a C₁₋₂₀ inclusive straight or branched hydrocarbon moiety having at least one carbon-carbon double bond by the removal of a single hydrogen molecule. Alkenyl groups include, for example, ethenyl (i.e., vinyl), methallyl, propenyl, butenyl, 1-methyl-2-buten-1-yl, pentenyl, hexenyl, octenyl, allenyl, butadienyl, styryl, acryl, and the like The term “alkenyl” is used interchangeably with the term “olefin” or “olefinic” herein.

As used herein, the term “alkynyl” refers to a monovalent group derived from a straight or branched C₁-C₂₀ hydrocarbon of a designed number of carbon atoms containing at least one carbon-carbon triple bond. Representative examples of“alkynyl” include ethynyl, 2-propynyl (propargyl), 1-propynyl, pentynyl, hexynyl, and heptynyl groups, and the like.

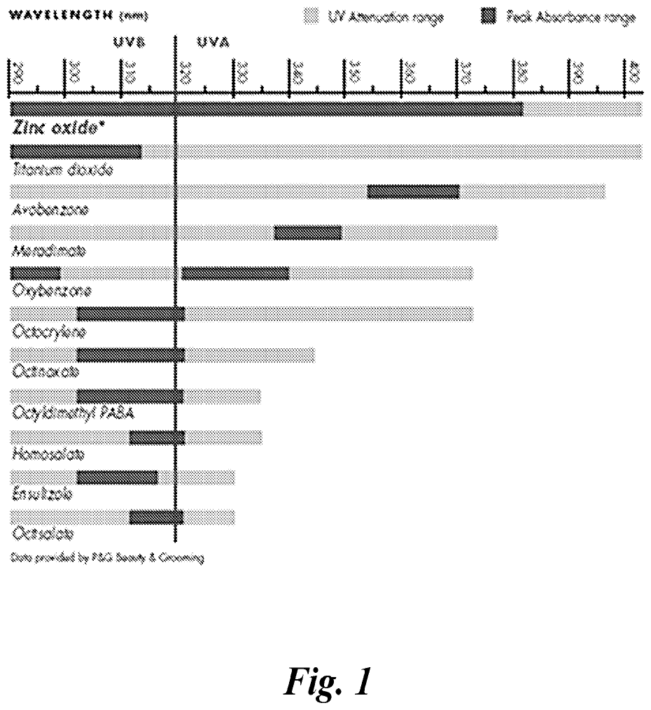

Based on their functionality and respective spectral absorptions, oxybenzone and para-aminobenzoic acid (PABA) derivatives were chosen as representative chromophores with which to demonstrate proof of concept. PABA derivatives, such as Padimate O (octyldimethyl para aminobenzoic acid), are strong UVB absorbers, while oxybenzone shows peak absorbances in both the UVA and UVB regions of the spectrum (see FIG. 1). The expectation is that they could be combined to obtain a film product providing strong absorbance between about 290 nm to about 340 nm, tailing out to 380 nm, thereby covering approximately 50-80% of the UV spectrum.

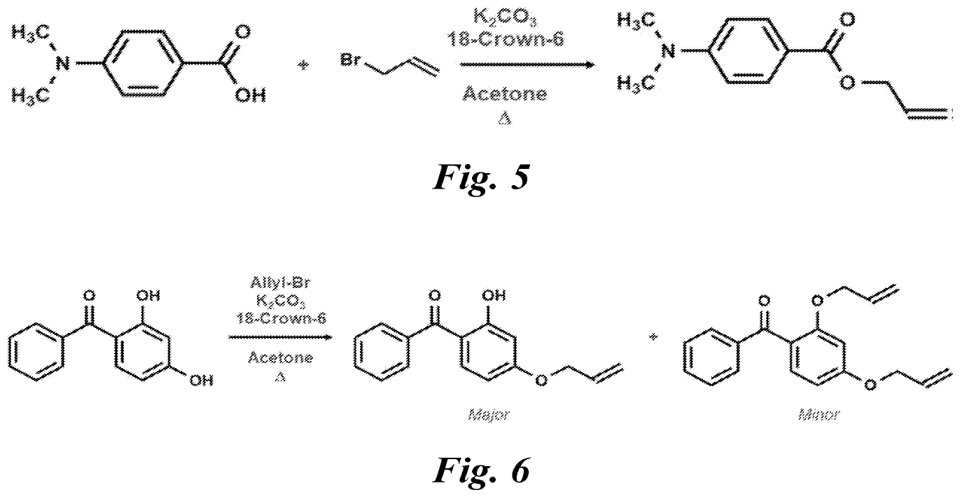

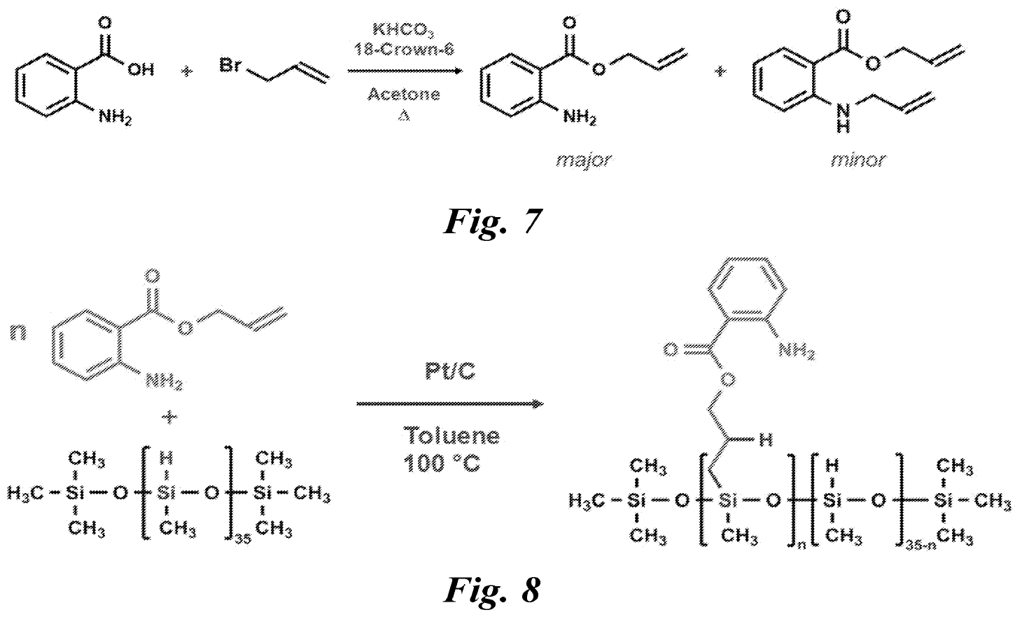

For the synthesis of the PABA derivative, 4-dimethylamino benzoic acid was reacted with allyl bromide under phase transfer conditions (K₂CO₃, 18-crown-6, acetone) as depicted in FIG. 5 to afford the desired allyl ester in quantitative yield without the need for purification.

For the synthesis of the oxybenzone derivative, starting from 2,4-dihydroxybenzophenone, attempts to selectively allylate the hydroxyl group in the 4-position using the same conditions used to make the PABA derivative resulted in mixtures of the mono- and di-functional adducts (FIG. 6). Although the targeted mono-functional adduct was formed as the major product it could not be isolated as a pure compound. Several attempts to selectively form the target compound under milder conditions (e.g., by using lower temperatures and weaker bases) proved equally futile. Ultimately, the pursuit of this benzophenone derivative was abandoned in favor of other UVA/UVB absorbers with potentially more straightforward synthesis/purification protocols.

Meradimate, an ester of 2-aminobenzoic acid the structure of which is shown in FIG. 2A, was subsequently selected as a suitable alternative. As can be seen in FIG. 1, even though meradimate has no significant UVB absorption and has a lower molar absorptivity than oxybenzone, its strong peak absorption just below 350 nm makes it a good choice as a representative UVA absorber. Moreover, its molecular similarity to PABA suggested that the chemistry described above might be effective in its synthesis. Interestingly, use of those, as well as a range of milder reaction conditions, unexpectedly and consistently yielded complex mixtures of products (FIG. 6). A detailed thin layer chromatography (TLC) and gas chromatography-mass spectrometry (GC-MS) investigation was undertaken to map the components of the mixture. Ultimately, the desired mono-reacted ester product was identified and determined to be sufficiently separated from the other components to be isolated by chromatography, which was subsequently used to purify the compound in good yield.

### 1.2.2 Conjugation of the Functionalized Chromophores to Oligomeric Silicone Scaffolds

The allyl-functional UV absorbers were then conjugated to reactive oligomeric silicone scaffolds, which would later act as the coupling partners and crosslinkers during polymerization and film formation. For this purpose, HMS-993, a commercially available poly(methylhydro)siloxane oligomer that has an average degree of oligomerization of 35 was chosen to act as the scaffold. The goal was to couple as many chromophores as possible to HMS-993 to achieve the highest loading of UV-absorber in the final film.

Initial attempts were made to react the poly(methylhydro)siloxane with 17 equivalents of allyl-2-aminobenzoate under hydrosilylation conditions, as shown in FIG. 8, in an effort to functionalize half of the available Si—H sites. It was found, however, that even with prolonged reaction times and additional portions of Pt catalyst, it was not possible to drive the reaction to completion (i.e., complete consumption of the allyl ester). Without wishing to be bound to any one particular theory, it was thought that this was due to steric crowding around the silicone backbone with each coupling event that shielded the remaining Si—H groups from reaction once a certain number had been consumed.

Reducing the number of equivalents of allyl ester from 17 to 12 (33% of the Si—H groups) did not change the outcome. When the number of equivalents was further reduced to 9 (25% of the Si—H groups), however, the reaction could be driven to the point where only traces of the starting allyl ester remained. Only by using 7 equivalent (20% of the Si—H groups) or less, could the complete coupling of all chromophores occur. The 20% loading worked equally well for attachment of the allyl-functional PABA chromophores (FIG. 9).

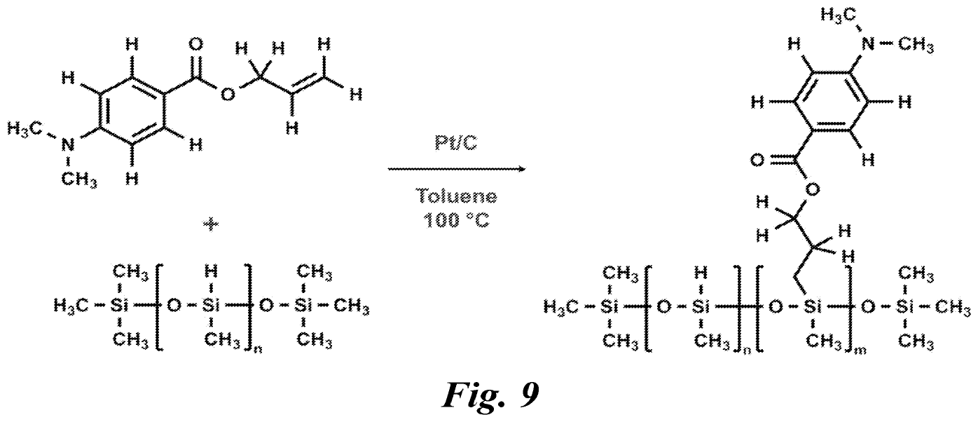

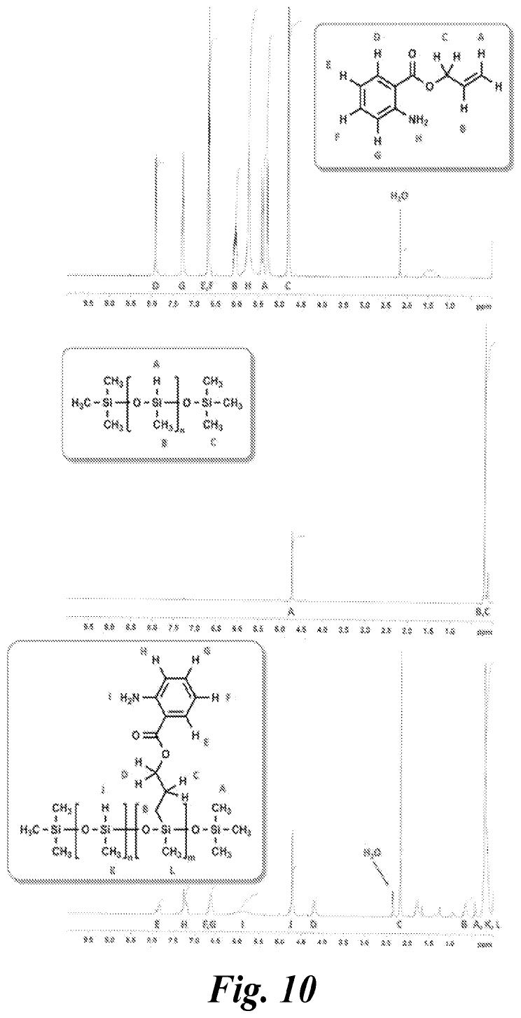

FIG. 10 shows the ¹H NMR spectra of the starting allyl-2-aminobenzoate, HMS-993, and the final product (with a 20 mol % loading), respectively. Comparison of the ¹H NMR spectra confirms that the product contains the chromophore, determined by the signals labeled D, E, G, H, and I, appearing between 5.7 and 8.0 ppm, as well as unreacted Si—H (4.8 ppm). By taking the ratio of the integrals of the two sets of peaks, the loading of meradimate chromophore was determined to be approximately 20 mol %. It is important to note the absence of any remaining vinyl resonances from the allylic groups from the starting ester (5.3-5.5 ppm and 6.0 ppm) in the product, indicating that they were successfully converted in the reaction.

1.2.3 Copolymerization of the Chromophore-Labeled Scaffolds into Films for Characterization

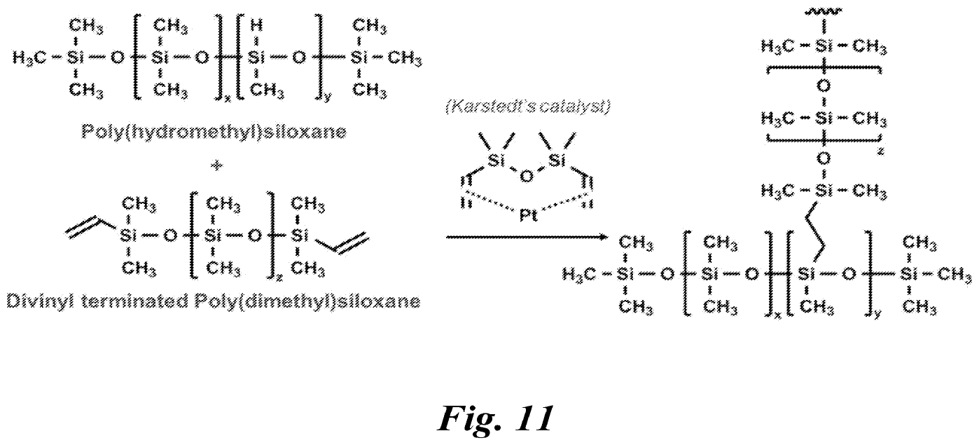

The next step was to copolymerize the newly synthesized UV chromophore-labeled silicone scaffolds into films by reacting them with complementary divinyl terminated poly(dimethyl)siloxane coupling partners. Hydrosilylation would again be used to affect this conversion, although under far more mild conditions than were used to prepare the labeled scaffolds using Karstedt's catalyst. In an effort to establish the reaction conditions, stoichiometry, and catalyst loadings without consuming the chromophore-labeled intermediates, conditions using unlabeled, commercially-available starting materials were considered (FIG. 11). A series of scouting and ladder studies were performed using HMS-993, the same poly(methylhydro)siloxane oligomer described above, and several divinyl-terminated poly(dimethyl)siloxanes (see Table 1).

TABLE 1
Summary of reactive silicone polymers and oligomers
used on the presently disclosed formulations

| | | Methylhydro- | | |
|---|---|---|---|---|
| Product | | siloxane | Viscosity | MW |
| Name | Polymer Type | (mole %) | (cSt) | (g/mol) |
| HMS-993 | Poly(methylhydro)siloxane | 100 | 30-45 | 2,100-2,400 |
| HMS-301 | Methylhydrosiloxane | 25-35 | 25-35 | 1,900-2,000 |
| | dimethylsiloxane copolymer | | | |
| DMS-V31 | Vinyl terminated PDMS | 0 | 1,000 | 28,000 |
| DMS-V41 | Vinyl terminated PDMS | 0 | 10,000 | 62,700 |
| DMS-V51 | Vinyl terminated PDMS | 0 | 100,000 | 140,000 |

Notes:
All polymers were purchased from Gelest, and data taken from Gelest Handbook of Reactive Silicones

Polymer films were formed by mixing HMS-993 with the vinyl-terminated PDMS polymers to a predetermined Si—H/vinyl ratio in a polypropylene beaker until homogeneous. The mixture was then applied to a non-stick coated substrate and combined with a small aliquot of catalyst solution before being drawn down and allowed to cure into a film. Although initial formulations contained only a single vinyl-terminated PDMS reactant, it was later found that compositions made up of mixtures of high and low molecular weight PDMS polymers were desirably elastic and robust. In particular, it was found that films derived from blends made of two parts DMS-V41 and one part DMS-V51 were sufficiently tough. That mixture was used for the remainder of this study.

Early experiments demonstrated the need for reinforcing fillers or resins, as even the best films would tear or crumble very easily during handling. Weaknesses in the film could be remedied by the addition of approximately 10 wt % fumed silica. The addition of fumed silica, however, was accompanied by two undesirable side effects. The most pronounced side effect was the opacification of the films. Although such opacification was anticipated (silica was eventually added to the pre-human subject testing formulations as a rheology modifier, reinforcement resin, and matting agent), the extent of opacification was higher than expected and was complicated by a second side effect—the introduction/generation of bubbles. All films made containing fumed silica contained bubbles.

It was unclear whether the bubbles formed during mixing or were generated during the cure by water-mediated reductive coupling of the Si—H groups, which generates hydrogen gas. It is conceivable that the source of the water could have been the fumed silica, which is very hygroscopic in spite of its hydrophobic surface treatment.

Unfortunately, the formation of the bubbles could not be completely avoided, even when the monomer mixture was degassed under vacuum prior to being drawn down. Their presence, together with the opacification resulting from the fumed silica, complicated the subsequent UV-vis analysis of the films. Bubble formation seemed to be worse at higher Si—H/vinyl ratios. Because of this observation and the fact films made at high Si—H/vinyl ratios cured exceedingly fast, sometimes as quickly as the catalyst solution was added, later polymer formulations were designed with Si—H/vinyl ratios of no more than 5:1, and more preferentially 2-3:1, using highly diluted catalyst solutions.

1.2.4 Formulate the Chromophore-Labeled Scaffolds into Two-Part Recipes for Direct Skin Application

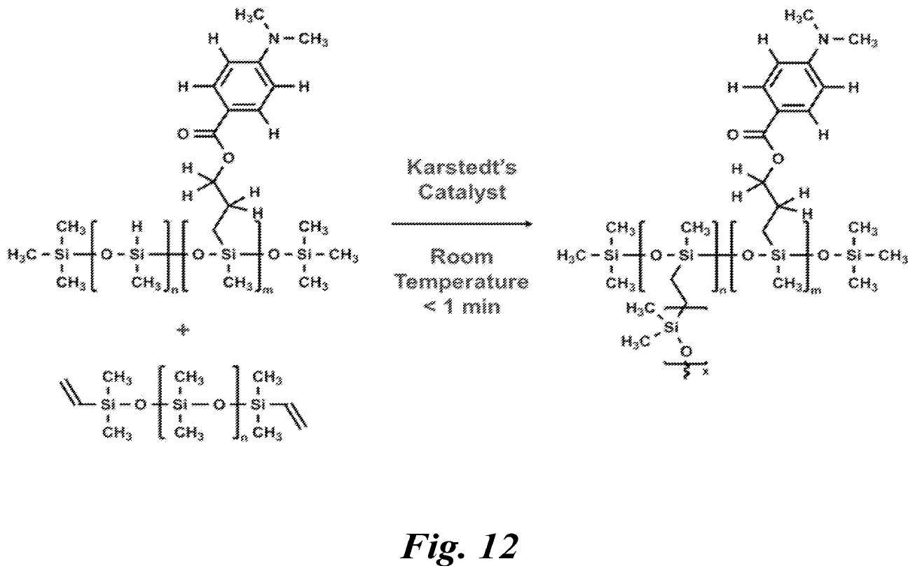

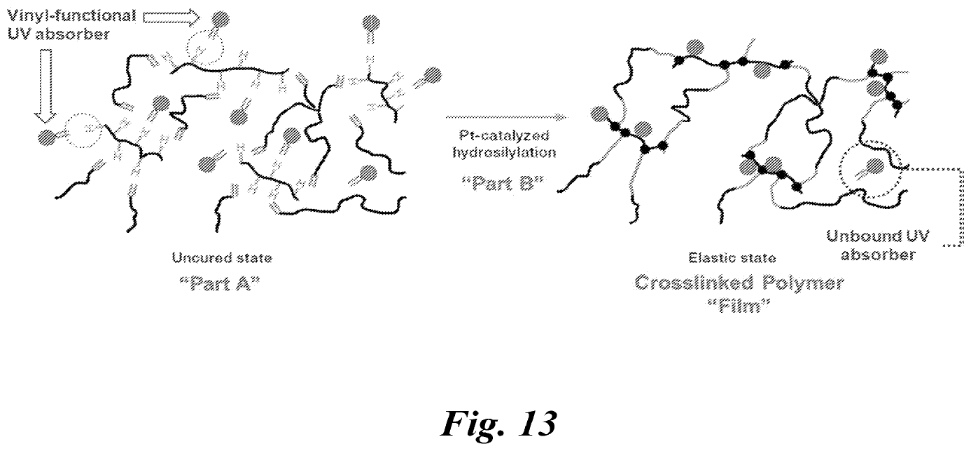

Having established some of the basic reaction parameters, films were prepared using the chromophore-labeled silicones. Initial attempts to prepare the PABA-functionalized film, according to FIG. 12, were only partially successful. The labeled and unlabeled reactants were able to be mixed, although the two were not very miscible, as evidenced by the opaque nature of the mixture (even before the addition of the fumed silica), captured in FIG. 13. Upon addition of the catalyst, the compositions cured too quickly (less than 1 minute) and did not produce satisfactory or bubble-free films.

When the experiment was repeated, the entire mass of the PABA-labeled silicone had unexpectedly turned into a solid, rubbery mass. Although the exact origin of this result was unclear, it was thought that it originated from oxidative instability of the chromophore, previously described in the literature. Shaath, 2007. Upon careful consideration, it was surmised that this fate could potentially befall almost any chromophore-labeled silicone intermediate that was made in this way, since most of them exhibit some form of photo- and/or oxidative instability and have the potential to produce radical intermediates. Accordingly, the approach of synthesizing chromophore-labeled UV absorbing silicone oligomers was abandoned in pursuit of other options less sensitive to degradation. Redesigning the system to eliminate this failure mechanism was determined to be crucial to the development of a shelf-stable, commercially-viable product.

### 1.2.5 One-Pot, Two-Step, Three Component Synthesis Strategy

It was recognized that the same chemical reaction, albeit under different conditions, was used in the synthesis of both the chromophore-labeled intermediates and in the film-forming polymerization. Whether both hydrosilylation reactions (covalent attachment of the chromophores and the silicone network formation) could be conducted simultaneously in a single reaction step, in which the chromophore-labeled film was produced directly by combining the allyl-functionalized UV absorbers, the poly(methylhydro)siloxane oligomers, and the vinyl-terminated PDMS in the presence of the Karstedt's catalyst was investigated. If successful, this strategy would greatly simplify the overall process by eliminating the need to prepare, store, and handle the labeled silicone intermediates. Furthermore, this modified approach also increases the final formulation latitude, since multiple allyl-functional chromophores could be easily combined in the final reaction step to broaden UV spectral coverage.

It is important to note, however, that this one-pot, two-step, three component strategy has a potential drawback relative to the previous approach—the possibility of unbound UV-chromophores in the final film, which creates the potential for diffusion into the body or the environment. Since the effects of steric congestion on the ability to drive the attachment of UV chromophores to the silicone scaffolds to completion was previously observed, it was expected that this could be exacerbated during a polymerization step in which the incoming chromophores were competing with much larger vinyl-functional polymers for reactive sites. One strategy to combat this issue was to increase the Si—H to vinyl ratio in the reaction to increase the number of sites available for attachment and minimizing the amount of UV absorbers used.

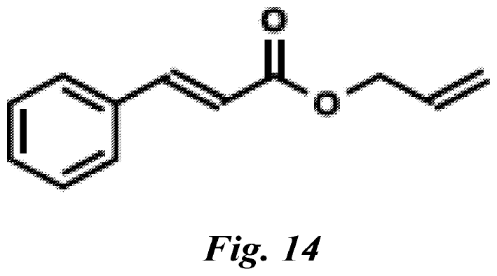

A commercially-available allyl ester was used to demonstrate the methodology. Allyl cinnamate (FIG. 14) was chosen as a model compound for this purpose as it is safe, inexpensive, and comprises the structural motif of octinoxate and related sunscreen components, although its absorption is blue shifted due to the absence of the electron-donating methoxy group on the aromatic ring. Interestingly, simple cinnamates esters of this type were amongst the first sunscreen chemicals employed in the earliest sunscreens developed in the late 1920s.

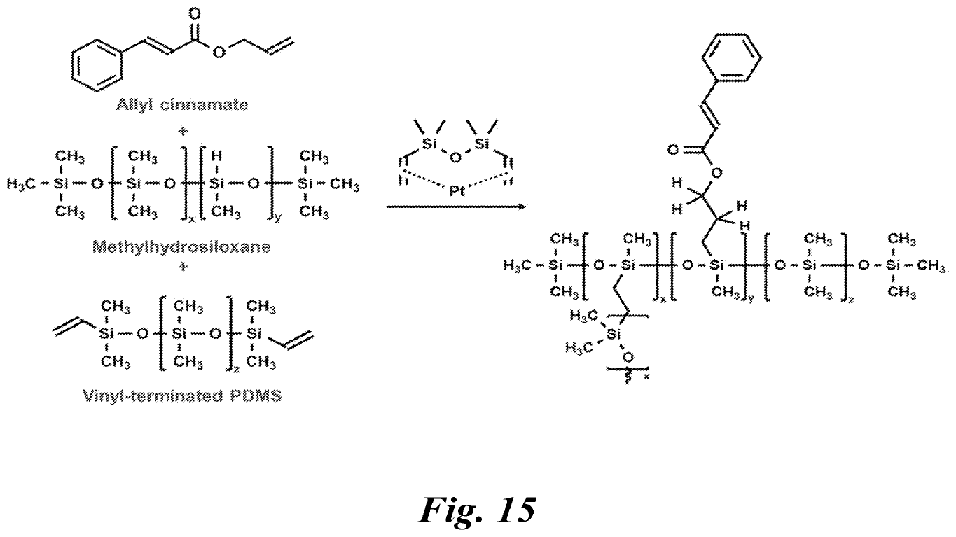

The three-component silicone film formation reactions initially pursued are depicted in FIG. 15. Although the reaction conditions are similar to those used hereinabove, several changes were made. Most importantly, the methylhydrosiloxane component from the homopolymer HMS-993 was switched to the copolymer HMS-301, which has approximately 70% lower Si—H content, to increase the film elasticity, and reduce the cure speed and the previously-noted propensity for bubble formation. In addition, this approach was supported with an increase in the dilution of the Karstedt's catalyst to 25 wt % to further manage the reaction kinetics. For these studies, the addition of fumed silica also was omitted to understand the cure kinetics and film appearance and properties in the absence of additional convoluting factors.

TABLE 2
Summary of cinnamate-labeled silicone films

| Component | 042619A^{d} | 042619B | 042619C | 042619D | 042619E |
|---|---|---|---|---|---|
| Vinyl- | 4.5 | 4.5 | 4.5 | 4.5 | 4.5 |
| terminated | | | | | |
| PDMS^{a} | | | | | |
| Methyl- | 1.8 | 1.8 | 1.8 | 1.8 | 2.7 |
| hydro- | | | | | |
| siloxane^{b} | | | | | |
| Allyl | 0.7 | 0.7 | 0.7 | 0.7 | 0.7 |
| cinnamate | | | | | |
| Pt catalyst | 100 | 200 | 400 | 300 | 300 |
| (ppm Pt)^{c} | | | | | |
| Si—H/vinyl | 2.0 | 2.0 | 2.0 | 4.0 | 3.0 |
| ratio | | | | | |

Notes:
^{a}A 2:1 (w/w) mixture of DMS-V41 and DMS-V51;
^{b}HMS-301, methyhydrosiloxane dimethylsiloxane copolymer;
^{c}SIP6830.3 diluted 1:3 (w/w) with EtOAc. Pt catalyst level is active Pt concentration based on total polymer and cinnamate mass;
^{d}Samples correspond to notebook number: AWF-######_

The compositions of initial reactions are described in Table 2. For these studies, the vinyl content (total vinyl PDMS and allyl cinnamate) was held constant while varying the Si—H and platinum levels. A high loading of cinnamate (10 wt %) was used to probe both the film formation under the most challenging conditions (i.e., high concentration of competing species) and the extent of chromophore binding. Based on concentrations of UV-absorbers in commercial sunscreens, it was anticipated that 10 wt % would be an upper concentration limit.

It was observed that the cure speed depended primarily on the Si—H to vinyl ratio. At low ratios, i.e., 2:1, the films required well over one hour to cure, with only slight acceleration observed at higher Pt loadings. Surprisingly, the films still exhibited some surface roughness and bubbles (presumably trapped during mixing) despite the absence of fumed silica. These detrimental features were again exacerbated at higher Si—H to vinyl ratios, disfavoring those conditions (Table 2, AWF-042619D and E) in spite of their desirably faster kinetics. Thus, the optimal conditions (i.e., those forming the smoothest films) identified in this study were reactions performed at a Si—H to vinyl ratio of 2:1, employing 200-300 ppm Pt catalyst. These films, however, which did not contain reinforcing resins of any kind (e.g., fumed silica), were still very weak and not easy to handle.

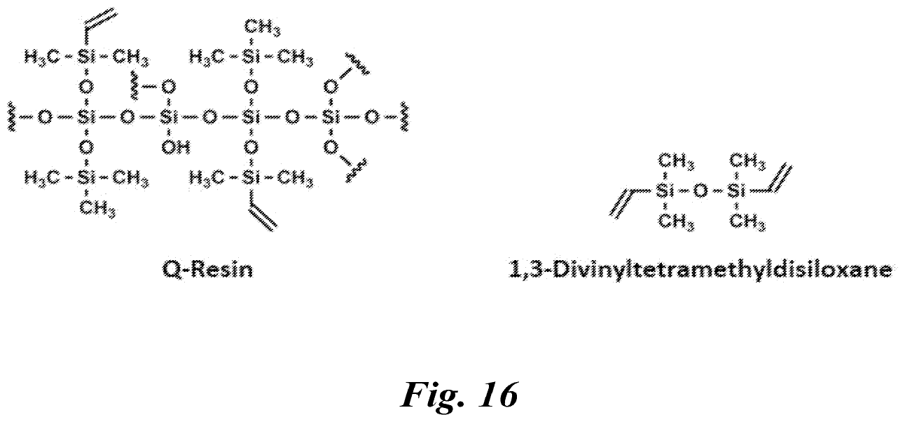

Building on this work, a final set of film studies was prepared. Recognizing the need to improve both the physical/mechanical properties of the films, as well as their quality, two new ingredients were added to the formulations—Q-resins and polymerization inhibitors (structures shown in FIG. 16). Q-Resins are high molecular weight silicone polymers that possesses a multiplicity of terminal vinyl groups that can participate in the hydrosilyation polymerization of the other monomers, acting as large, covalently bound reinforcing crosslinking centers within the network, performing similar function as fumed silica. Since they are refractive index matched with the rest of the silicone network, however, they improved toughness without impacting optical clarity.

TABLE 3
Summary of (un)labeled silicone films
prepared with Q-Resin and inhibitors

| Component^{a} | 061819B^{f} | 061819C | 061919D | 061919E | 061919F |
|---|---|---|---|---|---|
| Vinyl- | 7.5 | 7.5 | 5.4 | 6.0 | 7.5 |
| terminated | | | | | |
| PDMS^{b} (g) | | | | | |
| HMS-301 | 0.1 | 0.1 | 2.0 | 1.0 | 0.1 |
| (g) | | | | | |
| Q-Resin | 1.6 | 3.2 | 3.0 | 3.0 | 3.0 |
| (wt %)^{c} | | | | | |
| Allyl | — | — | 8.0 | 4.0 | — |
| cinnamate | | | | | |
| (wt %)^{d} | | | | | |
| ZnO (wt %)^{d} | — | — | — | — | 1.0 |
| Pt catalyst | 50 | 50 | 100 | 100 | 50 |
| (ppm Pt)^{e} | | | | | |
| Si—H/vinyl | 2.0 | 2.0 | 2.4 | 2.4 | 2.0 |
| ratio | | | | | |

Notes:
^{a}all samples contain 0.05 g 1,3-divinyltetramethyldisiloxane;
^{b}A 2:1 (w/w) mixture of DMS-V41 and DMS-V51;
^{c}wt % of active Q-Resin (VQM-146), relative total polymer mass;
^{d}wt % relative to total polymer mass;
^{e}SIP6830.3 diluted 1:7 (w/w) with EtOAc. Pt catalyst level is active Pt concentration based on total polymer and cinnamate mass;
^{f}Samples correspond to notebook number: AWF-######_

The second new ingredient added to the formulation was a polymerization inhibitor. Over the course of the film studies described above, it was difficult to strike the correct balance between film cure speed, appearance, and properties. Literature suggested that the inclusion of fugitive (volatile) inhibitors might provide the on/off switch that was needed by extending the working time of the silicone formulations and postponing cure until the inhibitor evaporates. Without wishing to be bound to any one particular theory, it was thought that incorporating such a feature could allow enough time to enable freeing of the entrapped air bubbles, producing defect-free films. 1,3-divinyltetramethyldisiloxane is a safe, low-cost, and volatile inhibitor of hydrosilylation reactions that can function in very low concentration. Ironically, it also serves as the ligand for Karstedt's catalyst, thereby it was already present in the present mixtures, albeit at much lower concentrations.

The experimental design of this film study is outlined in Table 3. In initial experiments, the effect of the inhibitor and Q-resin loading on the preparation of clear, unlabeled films was investigated. As described in the table, formulations 061819D and E were made using 1.6 and 3.2 wt % Q-resin, respectively. Both formulations contained two drops (50 mg) of added inhibitor. For these samples, a lower Pt catalyst levels (50 ppm) than had been used in earlier formulations was attempted to be used. Interestingly, control samples (not shown) prepared without the inhibitor cured so quickly that they solidified before the catalyst was completely mixed in. The inclusion of only 50 mg of inhibitor, however, completely suppressed the cure, allowing the components to be well mixed, applied to the substrates, and drawn down cleanly. Furthermore, the cure was sufficiently slowed allowing any entrained air bubbles time to escape. This, in turn, allowed the film to cure into a perfectly smooth, defect-free film upon drying overnight. The time to reach full cure was not noted, but was at least 30 minutes.

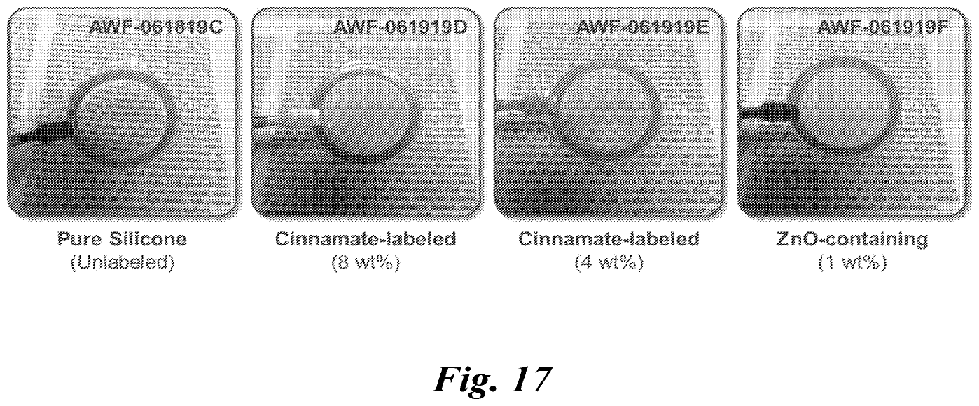

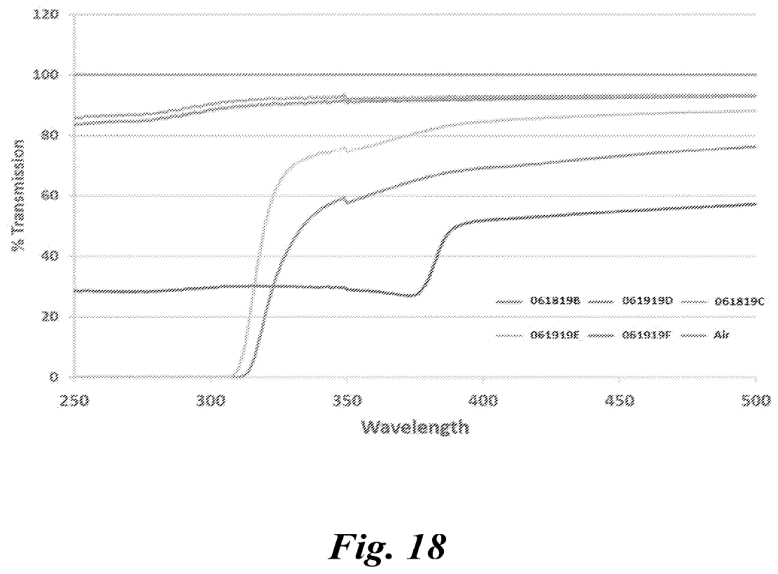

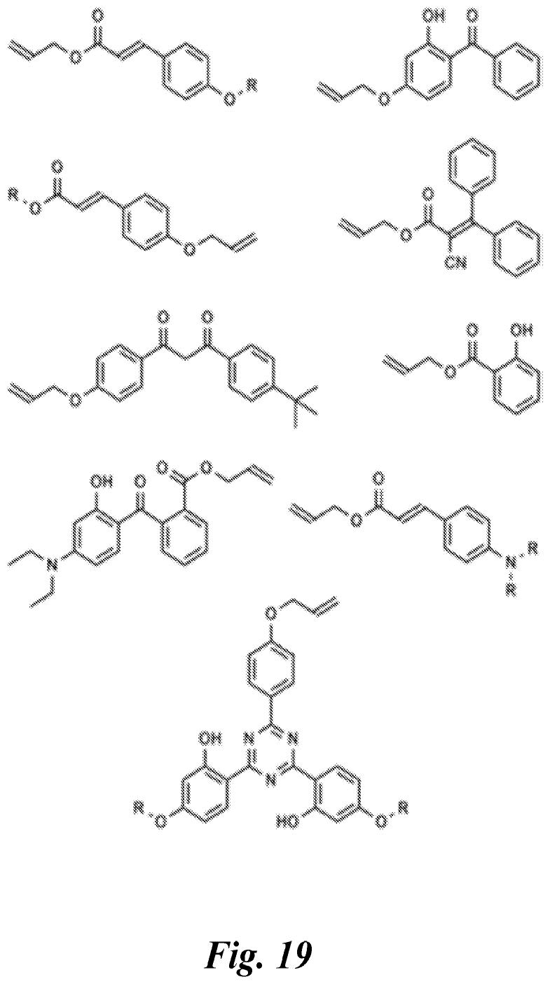

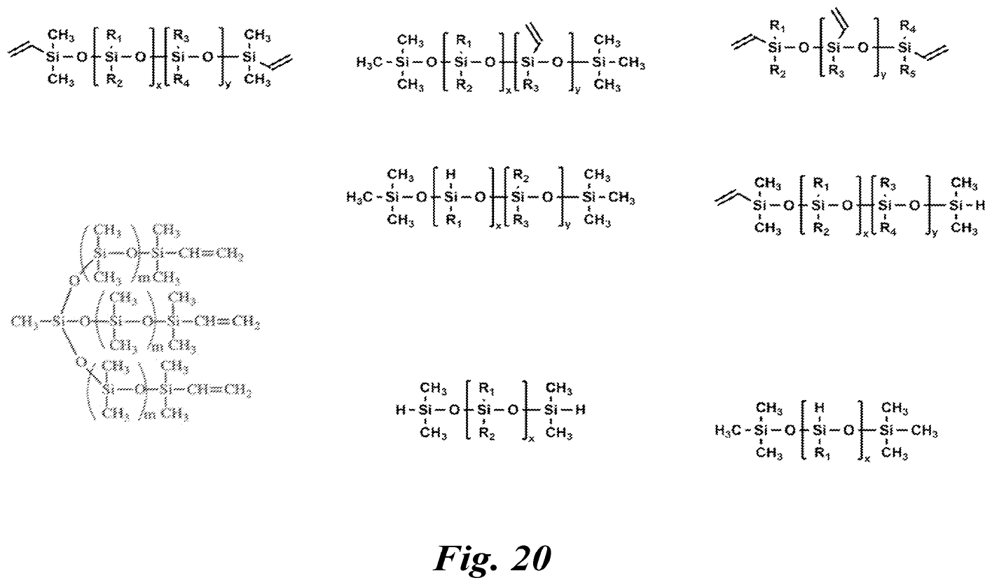

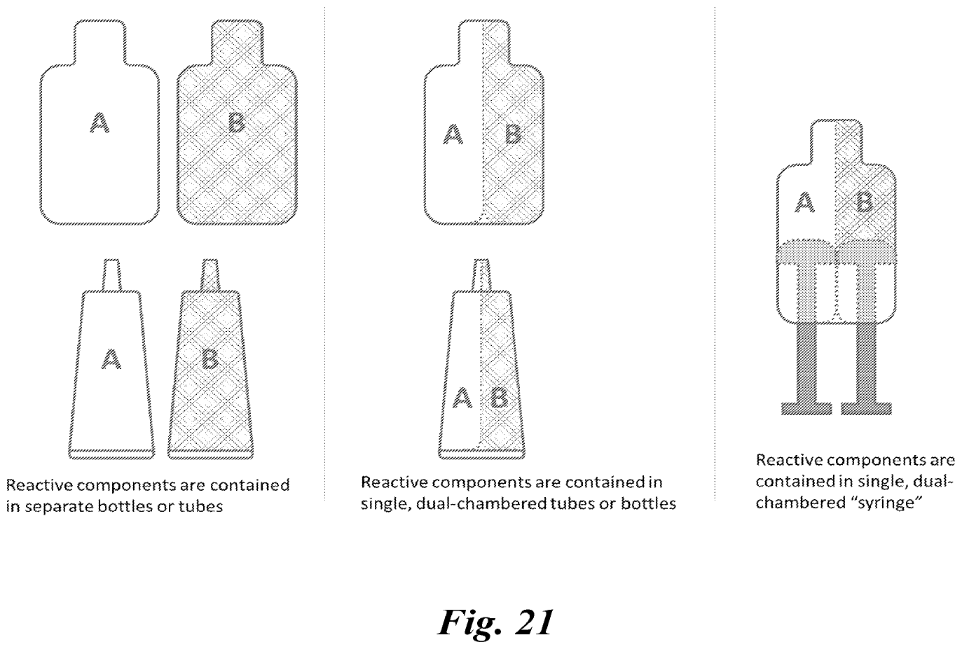

Once finally cured, the films were tough and elastic. They peeled from their substrates easily and intact. As expected, continued handling and stretching of the films revealed that those containing the higher level of Q-Resin (Table 3, AWF061819C) were slightly more robust. The modification to the formulation appeared to yield desirably smooth, tough, and clear films. The optical properties and quality of the film can be seen in photos in FIG. 17.

Encouraged by the results with the unlabeled films, cinnamate-functional films based on formulation AWF-061819C were prepared. For these experiments, two formulations with different cinnamate loadings were prepared: AWF-061919D and E, with 8 and 4 wt %, respectively. Additionally, the Si—H to vinyl ratio was increased slightly from 2.0 to 2.4, and the Pt catalyst loading doubled to accelerate film cure slightly and to provide more Si—H sites to improve chromophore attachment. Even with those modifications, the trace amount of inhibitor prevented premature curing, and allowed the film to form cleanly (see FIG. 17). Again, the time to reach full cure was not specifically noted, but exceeded 30 minutes. As seen in the photos, films of AWF-061919D, containing 8 wt % cinnamate were translucent, while those of AWF-061919E, with half the loading of cinnamate were nearly transparent. Again, without wishing to be bound to any one particular theory, it was thought that the opacity results from the chromophore-rich phase separated domains that form during mixing, which are locked in place during cure. This phenomenon would be expected to have important ramifications on the appearance of coating cured directly on the skin, where aesthetics are important.

Having established optimal film formation processes and properties with the one-pot, two-step, three-component approach using organic UV absorbers, a final experiment was performed to probe the possibility of incorporating dispersed inorganic sunscreen ingredients in the formulation. Since most commercially-available broad spectrum sunscreens contain a mixture of organic absorbers and inorganic pigments (e.g., ZnO or TiO₂), Sinrich, 2018; Nanoparticles in Sunscreen, it seemed plausible that a similar strategy would have to be adopted to achieve total protection across the entire UV spectrum.

To that end, one formulation, AWF-061919F, which contained 1 wt % ZnO, was prepared. It is important to note that in contrast to the ZnO grades typically used as sunscreen ingredients, which are usually small particle size (<100 nm) and hydrophobically surface treated to aid in formulation and water resistance, Hofmann et al., 2017, the particular grade of ZnO used (ZOCO103) had a particle size of approximately 270 nm and was not surface treated. In spite of differences with respect to commercial sunscreen grades, ZOCO103 dispersed readily in the mixture of other silicone ingredients. Most importantly, it did not interfere with the hydrosilylation curing/film formation process, and actually yielded very smooth, defect-free films. As seen in FIG. 17, the film of AWF-061919F was quite opaque, even at the low loading used. This opacity is due to the efficient visible light scattering by the large particle size pigment, which would not be expected from the smaller size grades typically used in sunblocks.

The films described in Table 3 were subsequently characterized by UV-vis spectroscopy to measure their ability to attenuate light (see FIG. 18). The transparent silicone films, AWF-061819B and C, transmit nearly all of the UV and visible light. In contrast, both films containing cinnamate absorbers, AWF-061919D and E, completely block the transmission of UV light below approximate 310 nm, where the cinnamate absorbance is strongest. The performance of AWF-061919E is particularly impressive, given its low absorber content, of 4 wt %, which is at least half of the typical loading of UV filter molecules used in typical commercial sunscreens. The decrease in visible light transmission of AWF-061919D can be ascribed to its increased opacity, relative to AWF-061919E, noted above. The single ZnO-containing film, AWF-061919F, exhibits strongly reduced transmission across the UV spectrum, out to approximate 380 nm, which is consistent with the pigments absorption band. Again, visible light is scattered and attenuated by the opacity described above. The fact that AWF-061919F does not block 100% of the UV light between 250-380 nm, is a result of the low surface area, large particle size ZnO used, and its low loading in the film.

### 1.3 Experimental

### 1.3.1 Materials

2,4-Dihydroxybenzophenone, allyl bromide, allyl cinnamate, and platinum on carbon (Pt/C, 5% Pt) were purchased from Sigma-Aldrich. 4-Dimethylaminobenzoic acid, 2-aminobenzoic acid (anthranilic acid), 18-crown-6, KHCO₃, K₂CO₃, acetone, ethyl acetate (EtOAc), and hexanes were purchased from Oakwood Chemicals. Divinyl terminated polysiloxanes (DMS-V31, DMS-V41, DMS-V51), poly(methylhydrosiloxane)s (HMS-993 and HMS-301), Karstedt's catalyst (SIP6830.3), 1,3-divinyltetramethyldisiloxane, fumed silica, Q-resin (VQM-146) were purchased from Gelest. Zinc oxide, particle size ≥270 nm (ZOCO103) was obtained from Zochem, Inc. All chemicals and reagents were used as received. The transmittance data was measured using an Agilent Cary 5000 UV-Vis-NIR spectrophotometer with an integrating sphere. The sample was placed at the entrance of the integrating sphere and light transmitted into the integrating sphere was collected.

1.3.2 4-Allyloxy-2-hydroxybenzophenone

A 125-mL round bottom flask was charged with 2,4-dihydroxybenzophenone (4.85 g, 0.022 mol), allyl bromide (2.6 g, 0.022 mol), KHCO₃ (4.53 g, 0.045 mol), 18-crown-6 (0.29 g, 0.001 mol), and acetone (40 mL). The flask was topped with a reflux condenser, and the contents blanketed with argon. The reaction mixture was heated to reflux (65° C.), for 5 hours, then allowed to cool to room temperature, with stirring for approximately 40 hours, during which time the color changed from bright yellow to burnt orange. The contents were then vacuum filtered to remove the salts, and the solvent evaporated in vacuo. The residue was taken up in EtOAc (200 mL) and extracted with 1M HCl (100 mL), 1M NaHCO₃ (2×100 mL), DI water (100 mL) and saturated aqueous NaCl (80 mL). After drying over MgSO₄, the solvent was stripped. The reaction showed a complex mixture of products that was not purified further.

1.3.3 Allyl-4-dimethylamino Benzoate

A 125-mL round bottom flask was charged with 4-dimethylaminobenzoic acid (4.5 g, 0.027 mol), allyl bromide (3.0 g, 0.025 mol), K₂CO₃ (6.86 g, 0.050 mol), 18-crown-6 (0.33 g, 0.001 mol), and acetone (50 mL). The flask was topped with a reflux condenser, and the contents blanketed with argon. The reaction mixture was heated to reflux (65° C.), for 3 hours, then allowed to cool to room temperature, with stirring, overnight. The contents were then filtered to remove the salts, and the solvent evaporated in vacuo. The residue was taken up in EtOAc (200 mL) and extracted with DI water (100 mL), 1M NaHCO₃ (3×80 mL), and saturated aqueous NaCl (80 mL). After drying over MgSO₄, the solvent was stripped, yielding the analytically pure light yellow oil (4.89 g, 96% yield).

1.3.4 Allyl-2-aminobenzoate

A 200 mL round bottom flask was charged with 2-amino benzoic acid (7.5 g, 0.055 mol), allyl bromide (6.0 g, 0.050 mol), KHCO₃ (10 g, 0.10 mol), 18-crown-6 (0.66 g, 0.002 mol), and acetone (100 mL). The flask was topped with a reflux condenser, and the contents blanketed with argon. The reaction mixture was heated to reflux (65° C.), for 16 hours. The contents were then filtered to remove the salts, and the solvent evaporated in vacuo. The residue was taken up in EtOAc (200 mL) and extracted with 1M HCl (100 mL), 1M NaHCO₃ (2×100 mL), DI water (100 mL) and saturated aqueous NaCl (80 mL). After drying over MgSO₄, the solvent was stripped. The product was isolated by column chromatography, eluting with a gradient of hexanes to 25:75 EtOAc:hexanes. (6.48 g, 72% yield).

### 1.3.5 Synthesis of the Meradimate-Labeled Silicone Oligomers

A 100-mL round bottom flask was charged with Pt/C (0.01 g) and toluene (1.5 mL). The flask was then capped with a rubber septum and purged with a continuous flow of argon gas, and immersed in an oil bath that was pre-heated to 95° C. Then a solution of HMS-993 (1.0 g, 0.0004 mol) and allyl-2-aminobenzoate (1.33 g, 0.007 mol), dissolved in toluene (3.5 mL) was added dropwise over approximately 25 minutes via syringe. Once the addition was complete, the rubber septum was replaced with a reflux condenser and the reaction temperature was increased to 100° C. and stirring continued overnight. After approximately 16 hours of reaction, the reaction was cooled to room temperature and filtered over a bed of Celite. After removal of the solvent in vacuo, the product was obtained as a pale yellow oil, which was used without further purification.

### 1.3.6 Synthesis of the PABA-Labeled Silicone Oligomers:

A 100-mL round bottom flask was charged with Pt/C (0.12 g) and toluene (20 mL). The flask was then capped with a rubber septum and purged with a continuous flow of argon gas, and immersed in an oil bath that was pre-heated to 95° C. Then a solution of HMS-993 (1.2 g, 0.0053 mol) and allyl-4-dimethylaminobenzoate (1.33 g, 0.037 mol), dissolved in toluene (40 mL) was added dropwise over approximately 30 minutes via syringe. Once the addition was complete, the rubber septum was replaced with a reflux condenser and the reaction temperature was increased to 100° C. and stirring continued overnight. After approximately 16 hours of reaction, the reaction was cooled to room temperature and filtered over a bed of Celite. After removal of the solvent in vacuo, the product was obtained as a pale yellow oil, which slowly crystallized, and which was used without further purification.

### 1.3.7 General Procedure for the Preparation of Silicone Polymer Films

Divinyl terminated poly(dimethylsiloxane)s were combined (typically DMS-V41 and DMS-V51 in a 2:1 weight ratio) in a 100-mL polypropylene (PP) beaker and mixed using a glass stir rod. The mixture was then evacuated to remove the air bubbles. The required portion of the mixture was then transferred to another PP beaker where it was combined with HMS-301 and an allyl functionalized chromophore (if used) in the appropriate amounts to achieve the targeted Si—H to vinyl ratio (typically 2:1 to 5:1). Addition components, such as fumed silica, Q-resin, and polymerization inhibitors, would be added (if used). Once all components were charged, they were mixed slowly, using a glass stir rod, for approximately 30-60 seconds to obtain a homogeneous mixture. To the mixture was then added a pre-made solution of Karstedt's catalyst in EtOAc (generally diluted 1:3 (25%) or 1:8 (12.5%) with solvent), via syringe, to the targeted Pt loading (50-100 ppm) based on total Si—H and vinyl monomer mass. The mixture was then mixed with a clean glass rod, which was used to transfer it into 500-micron thick fiber washers, or to silicon coated substrates for draw downs. The films were then allowed to cure, overnight, or until they had completely solidified.

### Example 2

The following procedures were used to prepare silicone films containing various types of covalently bound, non-covalently bound, and dispersed ultraviolet (UV) filtering compounds.

### 2.1 General Preparation of Silicone Films Comprising UV-Filtering Compounds

A 50-mL polypropylene beaker was charged with a mixture of vinyl-terminated polydimethylsiloxane (PMDS) (DMS-V41 and DMS-V51), silicon hydride-containing copolymers (HMS-301), vinyl-terminated Q-resin (VQM-146), and the UV-filtering compound(s) of interest (see Table 4). The contents were mixed for approximately 1-2 minutes using a glass stirring rod. Entrained air was then optionally removed by placing the beakers in a vacuum chamber at room temperature and 0 Torr for 5 minutes.

Divinyltetramethyl disiloxane was then added and slowly mixed in using a glass stirring rod for 1 minute before the addition of a solution of Karstedt's catalyst. The pre-polymer mixture was then slowly mixed for an additional 90 seconds before a portion was transferred to a circular stainless-steel mold (dimensions=1.0-inch internal diameter, 0.5-mm thickness). The thickness of the coatings was established by removing the excess pre-polymer mixture down to the level of the surface of the mold using a glass rod or doctor blade. The coatings were then allowed to dry under ambient conditions, until tack-free, prior to handling.

TABLE 4

| | UV Filtering Compound | | | | | | | |
|---|---|---|---|---|---|---|---|---|
| Example | OS | ODP | ON | OC | OB | M | AB | ZnO |
| 1 | 7.2 | — | — | — | — | — | — | — |
| 2 | — | 7.2 | — | — | — | — | — | — |
| 3 | — | — | 7.2 | — | — | — | — | — |
| 4 | — | — | — | 7.2 | — | — | — | — |
| 5 | — | — | — | — | 7.2 | — | — | — |
| 6 | — | — | — | — | — | 7.2 | — | — |
| 7 | — | — | — | — | — | — | 7.2 | — |
| 8 | — | — | — | 4.5 | — | — | 3.0 | — |
| 9 | 5 | — | — | 4.5 | 4.0 | — | 3.0 | — |
| 10 | — | — | — | — | — | — | 7.2 | 5.0 |
| 11 | — | — | — | 4.5 | — | — | 3.0 | 5.0 |
| Comp 1 | — | — | — | — | — | — | — | — |

Notes:
All amounts shown are expressed as weight percent (w/w) of UV filter in film;
OS = Octyl salate;
ODP = Octyl dimethyl PABA;
ON = Octinoxate;
OC = Octocrylene;
OB = Oxybenzone;
M = Meradimate;
AB = Avobenzone;
ZnO = Zinc oxide

Chemical structures of the representative UV-filtering compounds provided in Table 4 are provided immediately herein below:

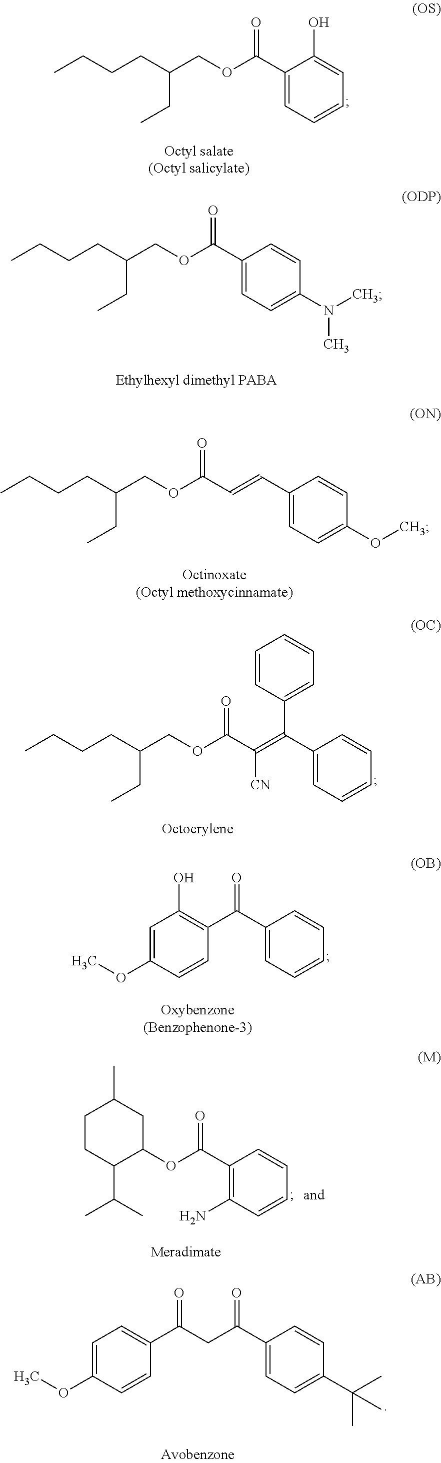

### 2.1.1 Preparation of Octyl Salate Containing Silicone Films—Example 1

### [Prophetic]

Octyl salate containing pre-polymer mixtures having compositions described below, and their resultant films were prepared according to the General Preparation of Silicone Films Comprising UV-Filtering Compounds described in Section 2.1.

TABLE 5

| | | wt % (based |
|---|---|---|
| Component | Mass (g) | on film mass) |
| Divinyl polydimethylsiloxane (DMS-V51) | 2.500 | |
| Divinyl polydimethylsiloxane (DMS-V41) | 5.000 | |
| Silicon hydride copolymer (HMS-301) | 0.100 | |
| Vinyl terminated Q-resin (VQM-146) | 0.228 | |
| UV filter (Octyl salate) | 0.610 | 7.2% |
| Karstedt's catalyst (0.3 wt % Pt) | 0.279 | |
| Divinyltetramethyl disiloxane | 0.080 | |

### 2.1.2 Example 2 (Preparation of Octyl Dimethyl-Para-Aminobenzoate (a.k.a Padimate O) Containing Silicone Films): [Prophetic]

Octyl para-dimethylaminobenzoate (ODP) containing pre-polymer mixtures and their resultant films were prepared according to the General Preparation of Silicone Films Comprising UV-Filtering Compounds, as in Example 1, except that octyl dimethyl-para-aminobenozate was used in place of octyl salate (OS).

### 2.1.3 Example 3 (Preparation of Octinoxate-Containing Silicone Films)

Octinoxate containing pre-polymer mixtures, and their resultant films were prepared according to the General Preparation of Silicone Films Comprising UV-Filtering Compounds, as in Example 1, except that Octinoxate (ON) was used in place of octyl salate (OS).

The same procedure was/could be used to prepare the remaining examples (4-11) in Table 4, the UV-filtering compound or combination described in the table. Note that Examples 4-11 are prophetic.

### 2.2 Preparation of Silicone Films Containing No UV Filters-Comparative Example 1

A pre-polymer mixture and its resultant film was prepared according to the General Preparation of Silicone Films Comprising UV-Filtering Compounds, as in Example 1, except that no UV-filtering compound was added.

TABLE 6

| Component | Mass (g) |
|---|---|
| Divinyl polydimethylsiloxane (DMS-V51) | 2.500 |
| Divinyl polydimethylsiloxane (DMS-V41) | 5.000 |
| Silicon hydride copolymer (HMS-301) | 0.100 |
| Vinyl terminated Q-resin (VQM-146) | 0.228 |
| Karstedt's catalyst (0.3 wt % Pt) | 0.279 |
| Divinyltetramethyl disiloxane | 0.080 |

### 2.3 Preparation of Silicone Film Compositions Comprising Reactive UV-Filtering Compounds

Table 7 shows silicone film compositions containing “reactive” UV-filtering compound, wherein the UV-filtering compounds contain reactive allyl functional groups that allow at least partial covalent attachment to the polymer backbone during the film cure.

TABLE 7

| | UV-Filtering Compound | | | | | | |
|---|---|---|---|---|---|---|---|
| | A₁- | A₁- | A₁- | A₁- | A₁- | A₁- | |
| Example | C | DP | ON | OC | OB | AB | ZnO |
| 12 | 7.2 | — | — | — | — | — | — |
| 13 | — | 7.2 | — | — | — | — | — |
| 14 | — | — | 7.2 | — | — | — | — |
| 15 | — | — | — | 7.2 | — | — | — |
| 16 | — | — | — | — | 7.2 | — | — |
| 17 | — | — | — | — | — | 7.2 | — |
| 18 | — | — | — | 4.0 | — | 3.0 | — |
| 19 | — | — | — | — | — | 7.2 | 5.0 |

Notes:
All amounts shown are expressed as weight percent (w/w) of UV-filtering compound in film;
A₁-C = Allyl cinnamate;
A₁-DP = Allyl dimethyl PABA;
A₁-ON = Allyl octinoxate;
A₁-OC = Allyl octocrylene;
A₁-OB = 4-Allyloxy oxybenzone;
A₁-AB = Allyloxy avobenzone;
ZnO = Zinc oxide 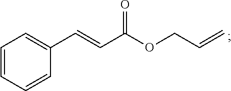
Allyl cinnamate (A₁-C) 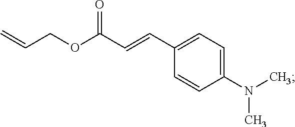 [SMILES: C=CCOC(=O)/C=C/c1ccc(N(C)C)cc1]
Allyl para-dimethylamino benzoate (A₁-DP) 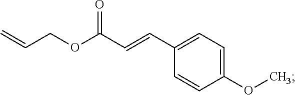 [SMILES: C=CCOC(=O)/C=C/c1ccc(OC)cc1]
Allyl octinoxate (A₁-ON) 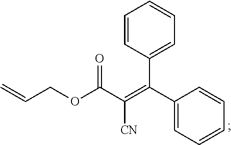 [SMILES: C=CCOC(=O)C(C#N)=C(c1ccccc1)c1ccccc1]
Allyl octocrylene (A₁-OC) 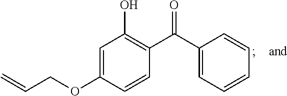 [SMILES: C=CCOc1ccc(C(=O)c2ccccc2)c(O)c1]
4-Allyloxy oxybenzone (A₁-OB) 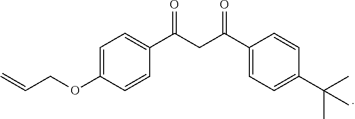 [SMILES: C=CCOc1ccc(C(=O)CC(=O)c2ccc(C(C)(C)C)cc2)cc1]
Allyloxy avobenzone (A₁-AB)

### 2.4 Preparation of Cinnamate-Containing Silicone Films Example 12

Cinnamate containing pre-polymer mixtures having compositions described below, and their resultant films were prepared according to the General Preparation of Silicone Films Comprising UV-Filtering Compounds described in Section 2.1.

TABLE 8

| | | wt % (based |
|---|---|---|
| Component | Mass (g) | on film mass) |
| Divinyl polydimethylsiloxane (DMS-V51) | 1.800 | |
| Divinyl polydimethylsiloxane (DMS-V41) | 3.600 | |
| Silicon hydride copolymer (HMS-301) | 2.000 | |
| Vinyl terminated Q-resin (VQM-146) | 0.220 | |
| UV filter (Allyl cinnamate A₁-C) | 0.592 | 7.2% |
| Karstedt's catalyst (0.3 wt % Pt) | 0.279 | |
| Divinyltetramethyl disiloxane | 0.080 | |

### 2.5 Preparation of Para-Dimethylaminobenzoate Containing Silicone Films—Example 13 [Prophetic]

para-Dimethylaminobenzoate containing pre-polymer mixtures and their resultant films were prepared according to the Preparation of Cinnamate-Containing Silicone Films as in Example 12, except that allyl para-dimethylaminobenzoate (A₁-DP) was used in place of allyl cinnamate (A₁-C).

The same procedure was/could be used to prepare the remaining examples (14-19) in Table 7 above, the UV-filtering compound or combination described in the table. Note that Examples 14-19 are prophetic.

### 2.6 Characterization of Representative Films

The films described above, including the comparative example were characterized for their UV attenuation properties and were found to attenuate the transmission of UV light through the film at wavelengths corresponding to their respective absorption spectra of their constituent UV-filtering compounds with peak reductions in transmission occurring at or near the absorption maxima of the UV-filtering compounds. In films containing multiple UV-filtering compounds, UV attention spanned the absorption of the multiple absorbers. Data are not shown here.

The films described hereinabove, including the comparative example were further characterized for their ability to retain their UV-filtering components under pseudo-physiological conditions with the potential to extract unbound or otherwise mobile UV-filtering compounds. In general, films were immersed in a gently agitated solution of artificial perspiration for 24 hours at room temperature. Following removal of the films, a small amount of dimethylsulfone was added to each extract solution before it was concentrated to dryness under vacuum (65° C./0 torr). The resulting residues were then taken up in DMSO-d₆ and analyzed by quantitative ¹H NMR against the added internal standard (dimethylsulfone). The method was validated by control samples, which were prepared by spiking the extracts derived from silicone films made without UV-filtering compounds, with known amounts of UV-filtering compounds immediately prior to concentration under vacuum. Subsequent analysis of the control samples shows that the UV-filtering compounds are not lost or degraded by the sample preparation and analytical methods. Importantly, quantitative ¹H NMR analysis of the samples derived from films containing UV-filtering compounds does not show the detectable presence of UV-filtering compounds for films comprised of non-covalently bound UV-filtering compounds, covalently bound UV-filtering compounds, or mixtures thereof.

### 2.7 General Procedure for the Assessment of Extractables and Leachables from UV Absorbers Containing Films

20-mL screw cap borosilicate vials were charged with 10 g of synthetic perspiration (purchased from Reagents). The appropriate silicone film (1-inch diameter, 0.30- to 0.50-mm thickness) was then added to the vials before they were capped and placed on an VWR® orbital platform mixer for 24 hours at room temperature. The films were then removed using freshly cleaned tweezers. Control solutions were then charged with the appropriate post-additive (see Table 9). All vials were charged with dimethylsulfone, and the contents then carefully concentrated to dryness using a rotary evaporator (0 torr, 65° C.) for 20-30 minutes. The vials were then capped prior to analysis. Approximately 1.0-1.5 mL of fresh DMSO-d₆ was then added to each vial, which was subsequently agitated using a vortex mixer for 15-30 seconds. Any undissolved salts were allowed to settle before transfer of the solutions to clean NMR tubes. Samples were then analyzed using a Bruker 400 MHz NMR spectrometer. Quantitative determination of the extracted UV filter(s) was then made by comparing the relative integration of distinct resonances derived from the UV filtering compound(s) against that of the internal standard (dimethylsulfone).

TABLE 9

| | Post-added | UV filter found |
|---|---|---|
| In vial | UV Filter | by ¹H NMR |
| Synthetic perspiration | None | n/d |
| Synthetic perspiration + | None | n/d |
| Comparative 1 film | | |
| Synthetic perspiration + | Octinoxate | 98% |
| Comparative 1 film | | |
| Synthetic perspiration + | None | n/d |
| Example 3 film | | |
| Synthetic perspiration + | None | n/d |
| Example 12 film | | |
| Synthetic perspiration + | None | n/d |
| Example 5 film | | |
| Synthetic perspiration + | None | n/d |
| Example 7 film | | |
| Synthetic perspiration + | None | n/d |
| Example 8 film | | |
| Synthetic perspiration + | None | n/d |
| Example 11 film | | |
| Synthetic perspiration + | None | n/d |
| Example 14 film | | |
| Synthetic perspiration + | None | n/d |
| Example 16 film | | |
| Synthetic perspiration + | None | n/d |
| Example 18 film | | |
| Synthetic perspiration + | None | n/d |
| Example 19 film | | |

Notes:
(1) UV filter found by ¹H NMR expressed as percent of compound found vs equimolar internal standard, assuming 100% extraction of the UV filter;
(2) n/d = not detected;
(3) Post-added UV filter added just prior to concentration on the extract;
(4) Comparative Example 1 film has no added UV filtering compounds;
(5) Example 3 film contains 7.2 wt % octinoxate;
(6) Example 12 film contains 7.2 wt % allyl cinnamate

Importantly, these experiments show that non-covalently bound and covalently bound UV filters in these types of silicone films are not extractable or leachable, in detectable amounts, by synthetic perspiration.

### REFERENCES

All publications, patent applications, patents, and other references mentioned in the specification are indicative of the level of those skilled in the art to which the presently disclosed subject mailer pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.
Shaath, N. A. Sunscreen Evolution in Sunscreens: Regulations and Commercial Development, 3rd ed.; Shaath, N. A. ed.; Informa Healthcare: London, 2011; pp 3-17.
EltaMD Home Page. https://eltamd.com/sun-care/ (accessed Jul. 31, 2019).
Matta, M. K. et al. Effect of Sunscreen Application Under Maximal Use Conditions on Plasma Concentration of Sunscreen Active Ingredients. J. Amer. Med. Assoc. 2019, 321, 2082-2091.
The Trouble with Ingredients in Sunscreens. https://www.ewg.org/sunscreen/report/the-trouble-with-sunscreen-chemicals/(accessed Jul. 31, 2019).
Wang. J. et al. Recent Advances on Endocrine Disrupting Effects of UV Filters. Int. J Environ. Res. Public Health 2016, 13(8), 782.
Schlumpf, M. et al. Exposure Patterns of UV Filters, Fragrance, Parabens, Phthalates, Organochlor Pesticides, PBDEs and PCBS in Human Milk: Correlations of UV Filters with Use of Cosmetics. Chemosphere 2010, 81(16), 1173-1183.
Downs, C. A. et al. Toxicopathological Effects of Sunscreen UV Filter Oxybenzone (Benzophenone-3), on Coral Planulae and Cultured Primary Cells and Its Environmental Contamination in Hawaii and the U.S. Virgin Islands. Arch. Environ. Contam. Toxicol. 2016, 70, 265.
Safe Sunscreen Council Home Page. https://safesunscreencouncil.org (accessed Jul. 31, 2019).
Hogue, C. Hawaii Lawmakers pass Ban on Sunscreen Chemicals. Chem. Eng. News [Online] May 8, 2018. https://cen.acs.org/policy/legislation-/Hawaii-lawmakers-pass-ban-sunscreen/96/web/2018/05 (accessed Jul. 31, 2019).
Yu, B. et al. Skin Compositions and Methods of Use Thereof. U.S. Pat. No. 8,691,202B2, Apr. 8, 2014.
Klykken, P. et al. Dow-Corning Product Literature, Silicone Film Forming Technologies for Healthcare Applications
Hofmann, R. J. et al. Fifty Years of Hydrosilylation in Polymer Science: A Review of Current Trends of Low-Cost Transition Metal and Metal-Free Catalysts, Non-Thermally Triggered Hydrosilylation Reactions, and Industrial Applications. Polymers 2017, 9, 534-571.
Wexler, A. et al. Partial Alkylation of Polyhydroxybenzophenones. U.S. Pat. No. 4,323,710, Apr. 6, 1982.
Shaath, N. A. SPF Boosters and Photostability of Ultraviolet Filters. Happi, 2007, 77-83.
Holser, R. A. et al. Preparation and Characterization of 4-Methoxy Cinnamoyl Glycerol. J. Amer. Oil Chem. Soc. 2008, 85, 347-351.
Sinrich, J. Everything You Need to Know About Zinc Oxide on Your Sunscreen. [Online], Jul. 2, 2018. https://www.dermstore.com/blog/what-is-zinc-oxide-sunscreen/ (accessed Jul. 31, 2019).
Nanoparticles in Sunscreen. https://www.ewg.org/sunscreen/report/nanoparticles-in-sunscreen/ (accessed Jul. 31, 2019).
U.S. Pat. No. 8,691,202 to Yu et al., for Skin Compositions and Methods of Use Thereof, issued Apr. 8, 2014, which is incorporated herein by reference in its entirety. See also,
U.S. Pat. No. 9,114,096 to Yu et al., for Skin Compositions and Methods of Use Thereof, issued Aug. 25, 2015;
U.S. Pat. No. 9,308,221 to Yu et al., for Skin Compositions and Methods of Use Thereof, issued Apr. 12, 2016;
U.S. Pat. No. 9,333,223 to Yu et al., for Compositions and Methods for Treating Conditions of Compromised Skin Barrier Function, issued May 10, 2016;
U.S. Pat. No. 9,511,034 to Garrett, for Method for Applying a Skin Treatment, issued Dec. 6, 2016;
International PCT Patent Application Publication No. WO2018052951A1 to Shienlin for Kits, Compositions and Methods for Wound Treatment and Management, published Mar. 22, 2018;
U.S. Pat. No. 10,543,161 to Farran et al., for Methods for Protecting and Improving the Appearance of Skin, issued Jan. 28, 2020; and
International PCT Patent Application Publication No. WO2020067582A1 to Akthakul et al., for Compositions and methods for application over skin, published Apr. 2, 2020.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A composition comprising one or more crosslinked polysiloxanes having one or more light-filtering compounds covalently or non-covalently bound thereto or otherwise associated therewith.

2. The composition of claim 1, wherein the one or more crosslinked polysiloxanes comprise one or more Si—H functional polymers or oligomers and at least one Si-vinyl functional polymer or oligomer selected from the group consisting of polymethylhydrosiloxane, polymethylhydrosiloxane copolymers, divinylpolysiloxane, vinylpolysiloxane, monovinyl, monohydride terminated polysiloxane, and combinations thereof.

3. The composition of claim 1 or claim 2, wherein the one or more crosslinked polysiloxanes comprise one or more polymethylhydrosiloxanes selected from the group consisting of:
(a) 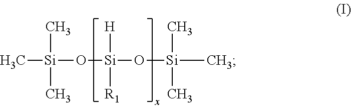
wherein:
R₁ is selected from the group consisting of hydrogen, C₁-C₂₀ alkyl, C₁-C₂₀ cycloalkyl, C₁-C₂₀ unsaturated alkyl, C₁-C₂₀ haloalkyl, hydroxylalkyl, alkoxyalkyl, carboxyalkyl, C₆-C₂₀ aryl, substituted aryl; and
x is an integer from 2 to 500;
(b) 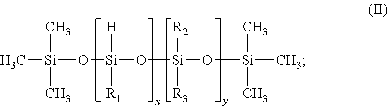
wherein:
R₁, R₂, R₃ are the same or different and are each selected from the group consisting of C₁-C₂₀ alkyl, C₁-C₂₀ cycloalkyl, C₁-C₂₀ unsaturated alkyl, C₁-C₂₀ haloalkyl, hydroxylalkyl, alkoxyalkyl, carboxyalkyl, C₆-C₂₀ aryl, and substituted aryl; and
x and y are each independently an integer from 1 to 500;
(c) 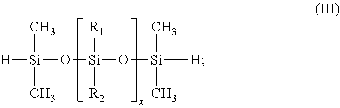
wherein:
R₁ and R₂ are the same or different and are each selected from the group consisting of hydrogen, C₁-C₂₀ alkyl, C₁-C₂₀ cycloalkyl, C₁-C₂₀ unsaturated alkyl, C₁-C₂₀ haloalkyl, hydroxylalkyl, alkoxyalkyl, carboxyalkyl, C₆-C₂₀ aryl, and substituted aryl; and
x is an integer from 1 to 1000; and
(d) 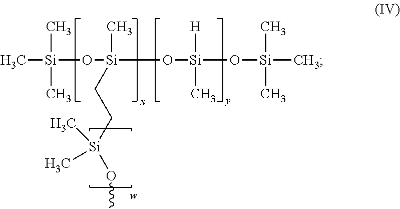
R₁ and R₂ are the same or different and are each selected from the group consisting of hydrogen, C₁-C₂₀ alkyl, C₁-C₂₀ cycloalkyl, C₁-C₂₀ unsaturated alkyl, C₁-C₂₀ haloalkyl, hydroxylalkyl, alkoxyalkyl, carboxyalkyl, C₆-C₂₀ aryl, and substituted aryl; and
x, y, and w are each independently an integer from 1 to 1000.

4. The composition of claim 1 or claim 2, wherein the one or more polysiloxanes comprise one or more divinylpolysiloxanes or vinylpolysiloxanes selected from the group consisting of:
(a) 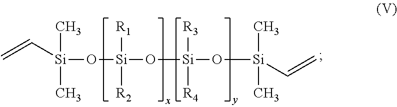
wherein:
R₁, R₂, R₃ and R₄ are the same or different and are selected from the group consisting of C₁-C₂₀ alkyl, C₁-C₂₀ cycloalkyl, C₁-C₂₀ unsaturated alkyl, C₁-C₂₀ haloalkyl, hydroxylalkyl, alkoxyalkyl, carboxyalkyl, C₆-C₂₀ aryl, and substituted aryl; and
x and y are each independently an integer from 1 to 500;
(b) 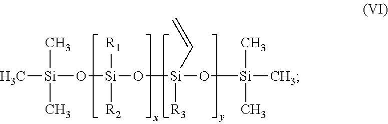
wherein:
R₁, R₂, and R₃ are the same or different and are each selected from the group consisting of C₁-C₂₀ alkyl, C₁-C₂₀ cycloalkyl, C₁-C₂₀ unsaturated alkyl, C₁-C₂₀ haloalkyl, hydroxylalkyl, alkoxyalkyl, carboxyalkyl, C₆-C₂₀ aryl, and substituted aryl; and
x is an integer from 0 to 500; and
y is an integer from 2 to 1000;
(c) 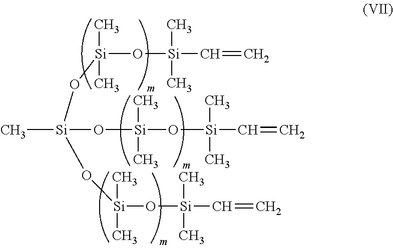
wherein each m is independently an integer from 1 to 100;
(d) 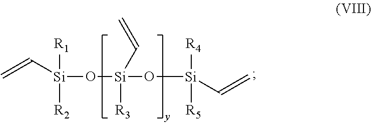
wherein;
₁, R₂, R₃, R₄, and R₅ are the same or different and are each selected from the group consisting of C₁-C₂₀ alkoxyl, C₁-C₂₀ alkyl, C₁-C₂₀ cycloalkyl, C₁-C₂₀ unsaturated alkyl, C₁-C₂₀ haloalkyl, hydroxylalkyl, alkoxyalkyl, carboxyalkyl, C₆-C₂₀ aryl, and substituted aryl; and
y is an integer from 1 to 1000; and
(e) 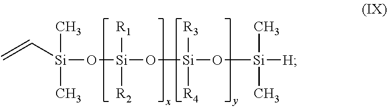
wherein:
R₁, R₂, R₃ and R₄ are the same or different and are selected from the group consisting of C₁-C₂₀ alkyl, C₁-C₂₀ cycloalkyl, C₁-C₂₀ unsaturated alkyl, C₁-C₂₀ haloalkyl, hydroxylalkyl, alkoxyalkyl, carboxyalkyl, C₆-C₂₀ aryl, and substituted aryl; and
x and y are each independently an integer from 1 to 500.

5. The composition of any one of claims 1-4, wherein the one or more polysiloxanes comprise a crosslinked polysiloxane network.

6. The composition of any one of claims 1-5, wherein the one or more light-filtering compounds comprise one or more organic light-filtering compounds.

7. The composition of claim 6, wherein the one or more organic light-filtering compounds comprise one or more organic UV-filtering compounds.

8. The composition of claim 7, wherein the one or more organic UV-filtering compounds is selected from the group consisting of: 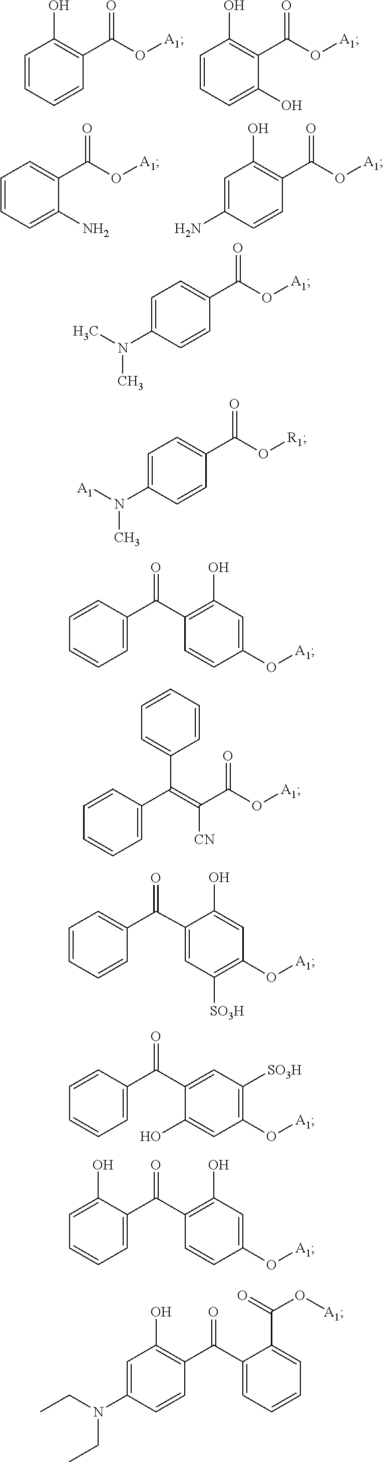 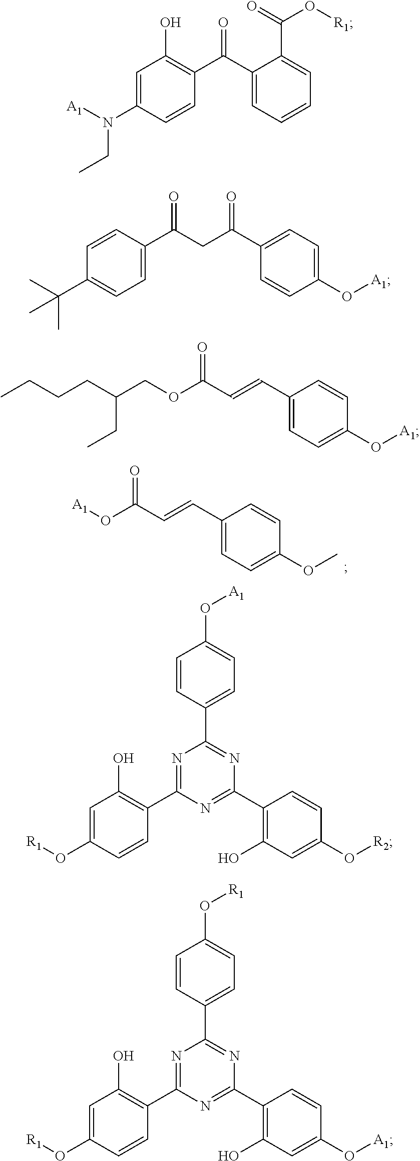 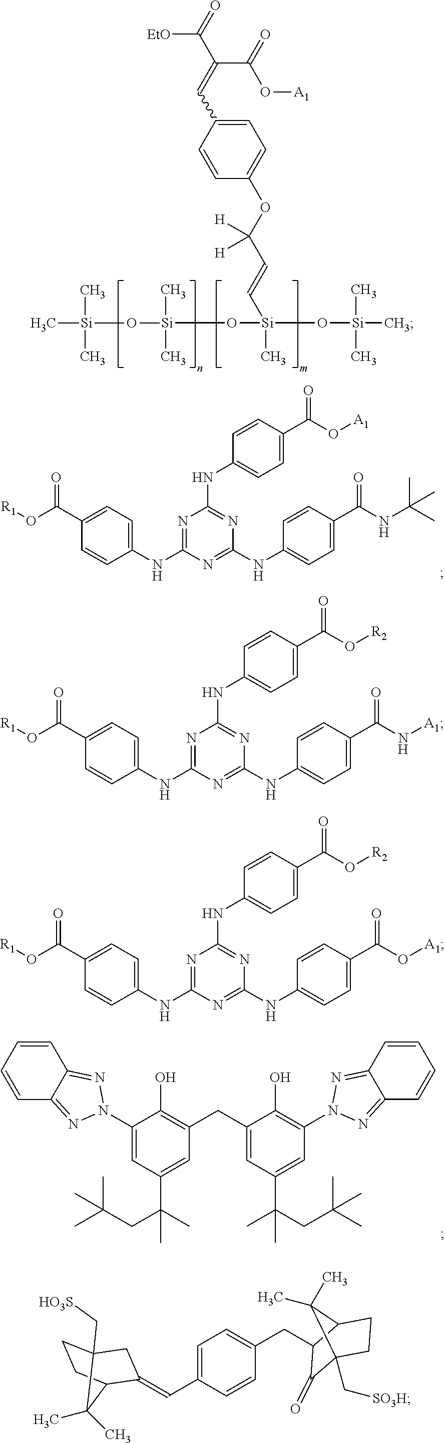 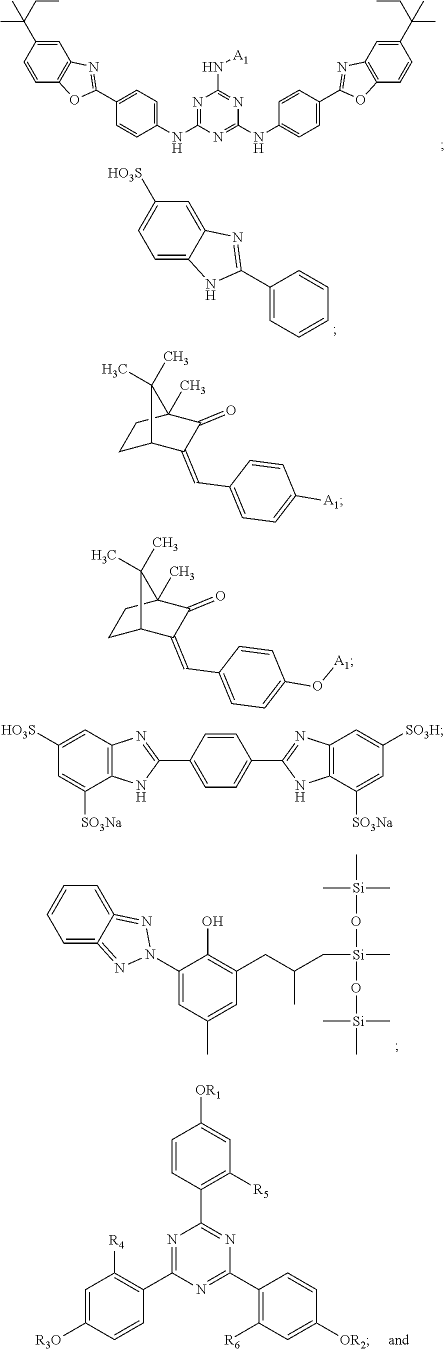 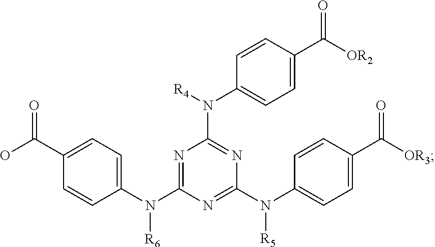
wherein:
A₁ is a moiety comprising at least one alkenyl or alkynyl group or A₁ comprises one or more of R₁, R₂, R₃, R₄, and R₅;
R₁, R₂, and R₃ are each independently selected from the group consisting of H, C₁-C₂₀ alkyl, aryl, and cycloalkyl; and
R₄, R₅, and R₆ are each independently selected from the group consisting of H, hydroxyl, C₁-C₂₀ alkoxyl, aryloxyl, and cycloalkoxyl.

9. The composition of claim 7, wherein the one or more organic UV-filtering compounds is selected from the group consisting of:

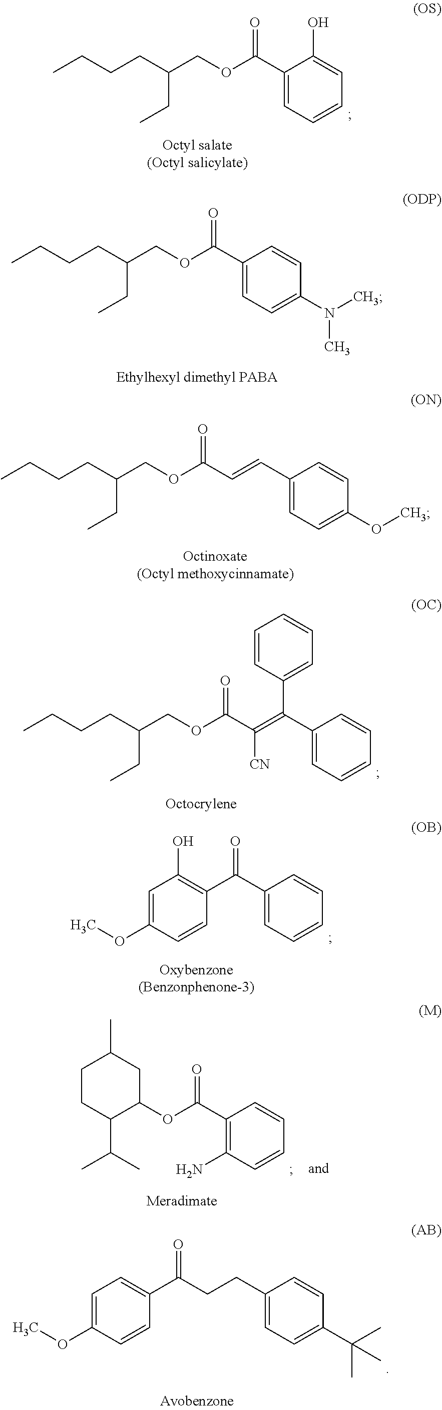

10. The composition of claim 7, wherein the one or more organic UV-filtering compounds comprises a reactive UV-filtering compound selected from the group consisting of:

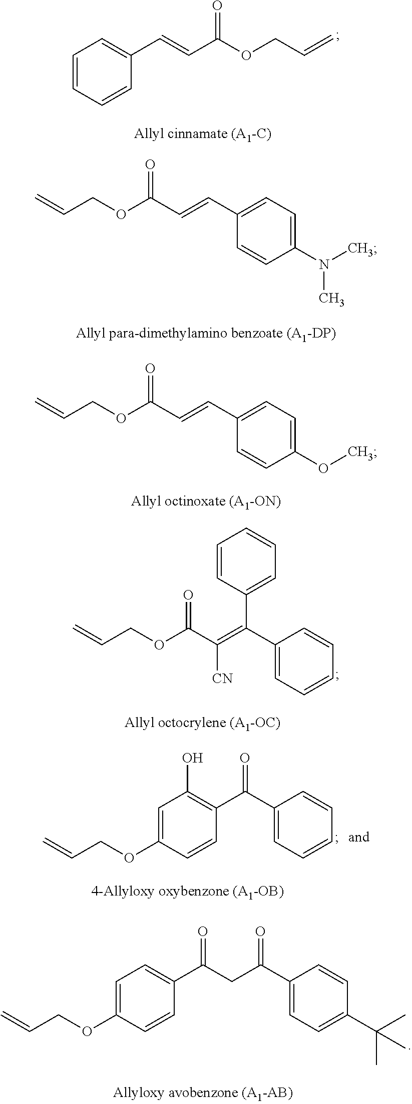

11. The composition of any one of claims 1-5, wherein the light-filtering compound comprises an inorganic light-filtering compound.

12. The composition of claim 11, wherein the inorganic light-filtering compound is selected from the group consisting of zinc oxide, titanium oxide, iron oxide, zirconium oxide, cerium oxide, and combinations thereof.

13. The composition of claim 1, further comprising a Q-resin.

14. The composition of claim 13, wherein the Q-resin is selected from the group consisting of: 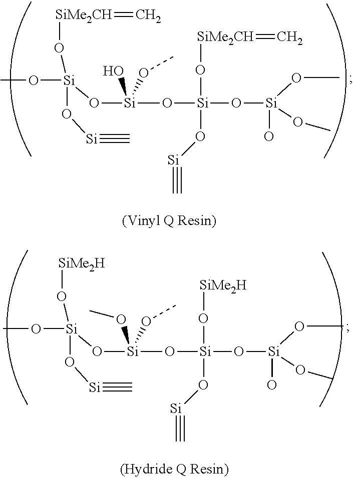
and combinations thereof.

15. The composition of any one of claims 1-14, further comprising an organic or inorganic reinforcing filler.

16. The composition of claim 15, wherein the inorganic reinforcing filler is selected from the group consisting of a clay, chalk, talc, calcite (CaCO₃), mica, barium sulfate, zirconium dioxide, zinc sulfide, zinc oxide, titanium dioxide, aluminum oxide, silica aluminates, calcium silicates, and a hydrophobically surface modified silica.

17. The composition of claim 16, wherein the reinforcing filler is selected from the group consisting of Al₂O₃ and SiO₂.

18. The composition of claim 16, wherein the hydrophobically surface modified silica is selected from the group consisting of fumed silica, hydrated silica, and anhydrous silica.

19. The composition of any of claims 1-18, wherein the one or more non-covalently bound light-filtering compounds are associated with the one or more crosslinked polysiloxanes by one or more of a hydrogen bond, a van der Waals interaction, π-stacking, a hydrophobic interaction, or combinations thereof.

20. A sunscreen comprising the composition of any one of claims 1-19.

21. A delivery device comprising the composition of any of claims 1-19 or components thereof.

22. The delivery device of claim 21, wherein the delivery device comprises two or more reactive components of the composition of any one of claims 1-19, wherein the two or more reactive components are contained in one or more of:
(a) separate bottles or tubes;
(b) a single, dual-chambered tube or bottle comprising a first chamber and a second chamber; and
(c) a single, dual-chambered syringe-like device comprising a first chamber and a second chamber.

23. The delivery device of claim 22, wherein the first chamber of the dual-chambered tube, bottle or syringe-like device comprises:
(a) a mixture of one or more polymethylhydrosiloxanes, one or more divinyl- or vinylpolysiloxanes, and one or more light-filtering compounds, whereas the second chamber comprises a catalyst and optionally one or more inhibitors; or
(b) a mixture of one or more polymethylhydrosiloxanes, one or more divinyl- or vinylpolysiloxanes, and one or more light-filtering compounds, whereas the second chamber comprises one or more divinyl- or vinylpolysiloxanes, a catalyst and optionally one or more inhibitors.

24. A method for preparing a composition of claim 1, the method comprising:
(a) providing or preparing one or more functionalized organic light-filtering compounds, non-functionalized organic light-filtering compounds, inorganic light-filtering compounds, or combinations thereof;
(b) providing or preparing one or more siloxane oligomers;
(c) contacting the one or more functionalized organic light-filtering compounds, non-functionalized light-filtering compounds, inorganic light-filtering compounds, or combinations thereof with the one or more siloxane oligomers to form one or more siloxane oligomers labeled with the one or more functionalized UV-filtering compounds or a mixture of the one or more siloxane oligomers with the one or more non-functionalized organic light-filtering compounds, the one or more inorganic light-filtering compounds, or combinations thereof;
(d) contacting the one or more siloxane oligomers labeled with the one or more functionalized UV-filtering compounds or a mixture of the one or more siloxane oligomers with the one or more non-functionalized organic light-filtering compounds, the one or more inorganic light-filtering compounds, or combinations thereof with one or more divinylpolysiloxanes, vinylpolysiloxanes, and combinations thereof in the presence of a catalyst to form a composition of claim 1.

25. The method of claim 24, wherein the one or more siloxane oligomers comprise one or more polymethylhydrosiloxanes of claim 3.

26. The method of claim 24, wherein the one or more divinylpolysiloxanes or vinylpolysiloxanes comprise the one or more divinylpolysiloxanes or vinylpolysiloxanes of claim 4.

27. The method of claim 24, further comprising adding a Q-resin to the composition.

28. The method of claim 27, wherein the Q-resin is a Q-resin of claim 14.

29. The method of claim 24, wherein the catalyst comprises a hydrosilylation catalyst.

30. The method of claim 29, wherein the hydrosilylation catalyst comprises a metal.

31. The method of claim 30, wherein the metal is selected from the group consisting of platinum, rhodium, tin, or a combination thereof.

32. The method of claim 31, wherein the metal is platinum and the hydrosilylation catalyst is selected from the group consisting of a platinum carbonyl cyclovinylmethylsiloxane complex, a platinum divinyltetramethyldisiloxane complex, a platinum cyclovinylmethylsiloxane complex, a platinum octanaldehyde/octanol complex, and combinations thereof.

33. The method of claim 31, wherein the metal is rhodium and the hydrosilylation catalyst is tris(dibutyl sulfide) rhodium trichloride.

34. The method of claim 31, wherein the metal is tin and the hydrosilylation catalyst is selected from the group consisting of tin II octanoate, tin II neodecanoate, dibutyltin diisooctylmaleate, di-n-butyl bis-(2,4pentanedionate)tin, di-n-butylbutoxychlorotin, dibutyltin dilaurate, dimethyltin dineodecanoate, dimethylhydroxy(oleate) tin, tin II oleate, and a combinations thereof.

35. The method of claim 24, further comprising adding an organic or inorganic reinforcing filler to the composition.

36. The method of claim 35, wherein the inorganic reinforcing filler is selected from the group consisting of a clay, chalk, talc, calcite (CaCO₃), mica, barium sulfate, zirconium dioxide, zinc sulfide, zinc oxide, titanium dioxide, aluminum oxide, silica aluminates, calcium silicates, and a surface-treated silica.

37. The method of claim 36, wherein the reinforcing filler is selected from the group consisting of Al₂O₃ and SiO₂.

38. The method of claim 36, wherein the surface-treated silica is selected from the group consisting of fumed silica, hydrated silica, and anhydrous silica.

39. A method of forming a composition of claim 1, the method comprising:
(a) combining one or more functionalized organic light-filtering compounds, non-functionalized organic light-filtering compounds, inorganic light-filtering compounds, or combinations thereof;
(b) one or more siloxane oligomers; and
(c) one or more divinylpolysiloxanes, vinylpolysiloxanes, monovinyl monohydride terminated polysiloxane and combinations thereof in the presence of a catalyst to form a composition of claim 1.

40. The method of claim 39, wherein the one or more siloxane oligomers comprise one or more polymethylhydrosiloxanes of claim 3.

41. The method of claim 39, wherein the one or more divinylpolysiloxanes or vinylpolysiloxanes comprise the one or more divinylpolysiloxanes or vinylpolysiloxanes of claim 4.

42. The method of claim 39, further comprising adding a Q-resin to the composition.

43. The method of claim 42, wherein the Q-resin is a Q-resin of claim 12.

44. The method of claim 39, wherein the catalyst comprises a hydrosilylation catalyst.

45. The method of claim 44, wherein the hydrosilylation catalyst comprises a metal.

46. The method of claim 45, wherein the metal is selected from the group consisting of platinum, rhodium, tin, or a combination thereof.

47. The method of claim 46, wherein the metal is platinum and the hydrosilylation catalyst is selected from the group consisting of a platinum carbonyl cyclovinylmethylsiloxane complex, a platinum divinyltetramethyldisiloxane complex, a platinum cyclovinylmethylsiloxane complex, a platinum octanaldehyde/octanol complex, and combinations thereof.

48. The method of claim 46, wherein the catalyst is Karstedt's catalyst:

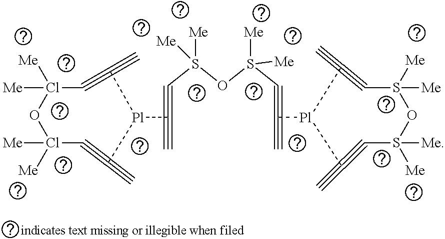

49. The method of claim 46, wherein the metal is rhodium and the hydrosilylation catalyst is tris(dibutyl sulfide) rhodium trichloride.

50. The method of claim 46, wherein the metal is tin and the hydrosilylation catalyst is selected from the group consisting of tin II octanoate, tin II neodecanoate, dibutyltin diisooctylmaleate, di-n-butyl bis-(2,4pentanedionate)tin, di-n-butylbutoxychlorotin, dibutyltin dilaurate, dimethyltin dineodecanoate, dimethylhydroxy(oleate) tin, tin II oleate, and a combinations thereof.

51. The method of claim 39, further comprising adding an organic or inorganic reinforcing filler to the composition.

52. The method of claim 51, wherein the inorganic reinforcing filler is selected from the group consisting of a clay, chalk, talc, calcite (CaCO₃), mica, barium sulfate, zirconium dioxide, zinc sulfide, zinc oxide, titanium dioxide, aluminum oxide, silica aluminates, calcium silicates, and a surface-treated silica.

53. The method of claim 52, wherein the reinforcing filler is selected from the group consisting of Al₂O₃ and SiO₂.

54. The method of claim 52, wherein the surface-treated silica is selected from the group consisting of fumed silica, hydrated silica, and anhydrous silica.

55. The method of any one of claims 39-54, comprising forming a film comprising a composition of claim 1.

56. The method of claim 55, further comprising forming a film on skin of a subject.

57. The method of claim 56, wherein the film is cured on the skin of the subject.

58. A method for attenuating or blocking an amount of radiation from penetrating skin of a subject, the method comprising applying to the skin of the subject at least one of:
(a) a film comprising a composition of any one of claims 1-19;
(b) a sunscreen of claim 20;
(c) a composition prepared by any one of claims 24-38;
(d) a composition prepared by any one of claims 39-54; or
(e) a film of any one of claims 55-57.
